(19) United States
(12) Patent Application Publication
(10) Pub. No.: US 2025/0099010 A1
(43) Pub. Date: Mar. 27, 2025

(54) SYSTEMS AND METHODS FOR PERFORMING EXERTION TESTING OF A PATIENT WEARING AN AMBULATORY MEDICAL DEVICE

(71) Applicant: ZOLL Medical Corporation, Chelmsford, MA (US)

(72) Inventors: Kent J. Volosin, Mars, PA (US); Uriel Weinstein, Mazkeret Batya (IL)

(21) Appl. No.: 18/728,262

(22) PCT Filed: Jan. 13, 2023
(86) PCT No.: PCT/US2023/060659

§ 371 Date: Jul. 11, 2024

Related U.S. Application Data
(60) Provisional application No. 63/301,558, filed on Jan. 21, 2022.

(51) Int. Cl. A61B 5/329; A61B 5/00; A61B 5/11; A61B 5/332; A61B 5/349

(52) U.S. Cl. CPC A61B 5/329; A61B 5/1118; A61B 5/332; A61B 5/349; A61B 5/4836; A61B 5/7285; A61B 2505/09

(57) ABSTRACT

An ambulatory medical device configured to assess a patient's physical condition is provided. The device includes externally worn ECG sensing electrodes configured to detect at least one cardiac electric signal of the patient, motion detectors configured to detect at least one motion signal indicative of patient movement, and a processor operably coupled to the sensing electrodes and the motion detectors. The processor is configured to receive, for the patient, initiating criterion corresponding to an ambulatory exertion test, wherein the initiating criterion specifies heartrate-dependent parameter requirements and/or activity level-dependent parameter requirements; continuously monitor a current heartrate-dependent parameter and/or a current activity level-dependent parameter of the patient; periodically determine whether the current heartrate-dependent parameter and/or the current activity level-dependent parameter satisfies the initiating criterion; invite the patient to participate in the ambulatory exertion test; and initiate, upon receiving patient input indicating acceptance of the invitation, the ambulatory exertion test.

### BACKGROUND

The present disclosure is directed to performing exertion testing of a cardiac rehabilitation patient.

Heart failure, if left untreated, can lead to certain life-threatening arrhythmias. Both atrial and ventricular arrhythmias are common in patients with heart failure. One of the deadliest cardiac arrhythmias is ventricular fibrillation, which occurs when normal, regular electrical impulses are replaced by irregular and rapid impulses, causing the heart muscle to stop normal contractions. Because the victim has no perceptible warning of the impending fibrillation, death often occurs before the necessary medical assistance can arrive. Other cardiac arrhythmias can include excessively slow heartrates known as bradycardia or excessively fast heartrates known as tachycardia. Cardiac arrest can occur when a patient in which various arrhythmias of the heart, such as ventricular fibrillation, ventricular tachycardia, pulseless electrical activity (PEA), and asystole (heart stops all electrical activity), result in the heart providing insufficient levels of blood flow to the brain and other vital organs for the support of life. It is generally useful to monitor heart failure patients to assess heart failure symptoms early and provide interventional therapies as soon as possible.

Patients who are at risk, have been hospitalized for, or otherwise are suffering from, adverse heart conditions can be prescribed a wearable cardiac monitoring and/or treatment device. In addition to the wearable device, the patient can also be given a battery charger and a set of rechargeable batteries. As the wearable device is generally prescribed for continuous or near-continuous use (e.g., only to be removed when bathing), the patient wears the device during all daily activities such as walking, sitting, climbing stairs, resting or sleeping, and other similar daily activities. During these activities, information about the patient can be recorded or otherwise observed for further analysis.

### SUMMARY

In an example, an ambulatory externally worn cardiac device configured to automatically assess an ambulatory patient's physical condition is provided. The device includes one or more externally worn ECG sensing electrodes configured to detect at least one cardiac electric signal of the ambulatory patient, one or more motion detectors configured to detect at least one motion signal indicative of patient movement, and at least one processor operably coupled to the one or more externally worn sensing electrodes and the one or more motion detectors. The at least one processor is configured to receive, for the ambulatory patient wearing the device, initiating criterion corresponding to an ambulatory exertion test, wherein the initiating criterion specifies heartrate-dependent parameter requirements and/or activity level-dependent parameter requirements, continuously monitor a current heartrate-dependent parameter and/or a current activity level-dependent parameter of the ambulatory patient, periodically determine whether the monitored current heartrate-dependent parameter and/or the current activity level-dependent parameter satisfies the initiating criterion for the ambulatory exertion test, invite, via a user interface prompt, the ambulatory patient to participate in the ambulatory exertion test, and initiate, upon receiving patient input indicating acceptance of the invitation, the ambulatory exertion test.

Implementations of the ambulatory externally worn cardiac device configured to automatically assess an ambulatory patient's physical condition can include one or more of the following features.

In examples of the ambulatory externally worn cardiac device, the heartrate-dependent parameter can include one or more of heartrate, energy exerted by the ambulatory patient, work performed by the ambulatory patient, heartrate variability, premature ventricular contraction burden or counts, atrial fibrillation burden, pauses, heartrate turbulence, QRS height, QRS width, changes in ECG morphology, cosine R-T, QT interval, QT variability, T-wave width, T-wave alternans, T-wave amplitude, T-wave variability, R-wave amplitude, and ST segment changes.

In examples of the ambulatory externally worn cardiac device, the current activity level-dependent parameter can include one or more of activity type, activity level, activity duration, energy exerted by the ambulatory patient, and work performed by the ambulatory patient.

In examples of the ambulatory externally worn cardiac device, the at least one processor can be further configured to receive a prescription for an ambulatory exertion test for the ambulatory patient from a healthcare provided associated with the ambulatory patient, the prescription including exertion test initiating criteria. In some examples, the at least one processor can be further configured to periodically determine whether the monitored current heartrate-dependent parameter and/or the current activity level-dependent parameter satisfies the exertion test initiating criteria, invite, via a user interface prompt, the ambulatory patient to participate in the ambulatory exertion test, and initiate, upon receiving patient input indicating acceptance of the invitation, the ambulatory exertion test. In some examples, the ambulatory exertion test can include a physical test directed to the ambulatory patient and is configured to prompt the ambulatory patient to perform a particular activity for a predetermined period of time. In some examples, the physical test can include a walking test, a jogging test, a running test, and/or a stair climbing test. In some examples, the predetermined period of time can include at least one of one minute, two minutes, five minutes, six minutes, ten minutes, fifteen minutes, twenty minutes, and/or thirty minutes.

In examples of the ambulatory externally worn cardiac device, the initiating criterion further can include monitoring the ambulatory patient performing a motion activity for a minimum period of time as determined for the ambulatory patient. In some examples, the motion activity can include walking, running, and/or climbing stairs. In some examples, the minimum period of time can include thirty seconds, one minute, two minutes, three minutes, five minutes, and/or ten minutes. In some examples, the ambulatory exertion test can include a physical test directed to the ambulatory patient and is configured to prompt the ambulatory patient to perform a particular activity for a predetermined period of time. In some examples, the physical test can include a walking test, a jogging test, a running test, and/or a stair climbing test. In some examples, the predetermined period of time can include at least one of one minute, two minutes, five minutes, six minutes, ten minutes, fifteen minutes, twenty minutes, and/or thirty minutes.

In examples of the ambulatory externally worn cardiac device, the at least one processor can be further configured to monitor the ambulatory patient during the ambulatory exertion test, determine if the ambulatory patient is satisfying at least one sustaining criterion during the ambulatory exertion test, the at least one sustaining criterion indicating whether the ambulatory patient is satisfactorily completing the ambulatory exertion test, and terminate the ambulatory exertion test if the ambulatory patient does not satisfy the at least one sustaining criterion. In some examples, the at least one sustaining criterion can include a minimum level of activity to be maintained by the ambulatory patient during the ambulatory exertion test and/or a target heart range for the ambulatory patient during the ambulatory exertion test.

In examples of the ambulatory externally worn cardiac device, the at least one processor can be further configured to monitor the ambulatory patient during the ambulatory exertion test, determine that the ambulatory patient has satisfied at least one terminating criterion for the ambulatory exertion test, provide an indication to the ambulatory patient that the ambulatory patient has completed the ambulatory exertion test, and terminate the ambulatory exertion test. In some examples, to terminate the ambulatory exertion test can include to generate a report including patient performance information as measured during the ambulatory exertion test. In some examples, the at least one terminating criterion can include a total distance walked, a total distance run, a total number of stairs climbed, and/or a total time elapsed.

In examples of the ambulatory externally worn cardiac device, the at least one processor can be further configured to monitor the cardiac electrical signal and the at least one motion signal for the ambulatory patient during the ambulatory exertion test and determine patient performance information for the ambulatory patient during the ambulatory exertion test. In some examples, at least one processor can be further configured to output at least one performance message to the ambulatory patient during the ambulatory exertion test, the performance message including one or more of an indication to increase pace, an indication to decrease pace, an indication to maintain current pace, an indication of time remaining, and/or an indication of elapsed time. In some examples, the at least one processor can be further configured to output at least a portion of the performance information to the ambulatory patient during the ambulatory exertion test, the performance information including one or more of total steps taken during the ambulatory exertion test, total distance covered during the ambulatory exertion test, total stairs climbed/descended during the ambulatory exertion test, average heartrate during the ambulatory exertion test, maximum heartrate during the ambulatory exertion test, and/or minimum heartrate during the ambulatory exertion test.

In examples of the ambulatory externally worn cardiac device, the at least one processor can be further configured to monitor cardiac activity for the ambulatory patient during the ambulatory exertion test based upon the at least cardiac electric signal, determine if the ambulatory patient is experiencing a cardiac arrhythmia during the ambulatory exertion test, and terminate the ambulatory exertion test if the ambulatory patient experiences a cardiac arrhythmia.

In examples of the ambulatory externally worn cardiac device, the at least one processor can be further configured to monitor current motion activity for the ambulatory patient during the ambulatory exertion test based upon the at least one motion signal, determine if the ambulatory patient is experiencing a fall event during the ambulatory exertion test, and terminate the ambulatory exertion test if the ambulatory patient experiences a fall event.

In examples of the ambulatory externally worn cardiac device, the at least one processor can be further configured to monitor the ambulatory patient during a baseline ambulatory exertion test and determine baseline performance information for the ambulatory patient based upon the baseline ambulatory exertion test. In some examples, the at least one processor can be further configured to monitor the ambulatory patient during the ambulatory exertion test, determine if the ambulatory patient is satisfying at least one sustaining criterion during the ambulatory exertion test, the at least one sustaining criterion being based upon the baseline ambulatory exertion test and indicating whether the ambulatory patient is satisfactorily completing the ambulatory exertion test, and terminate the ambulatory exertion test if the ambulatory patient does not satisfy the at least one sustaining criterion. In some examples, the at least one processor can be further configured to generate patient performance information for the ambulatory patient upon completion of the ambulatory exertion test, compare the ambulatory patient performance information to the baseline performance information to produce comparison exertion test information, and determine at least one trend for the ambulatory patient based upon the comparison exertion test information, the at least one trend indicating a change in an overall cardiac condition of the ambulatory patient.

In examples of the ambulatory externally worn cardiac device, the at least one processor can be further configured to administer a plurality of ambulatory exertion tests to the ambulatory patient, record patient performance information for each of the plurality of ambulatory exertion tests to generate historical patient performance information, administer a new ambulatory exertion test to the ambulatory patient, record updated patient performance information for the new ambulatory exertion test, compare the updated patient performance information to the historic patient performance information to produce comparison exertion test information, and determine at least one trend for the ambulatory patient based upon the comparison exertion test information, the at least one trend indicating a change in an overall cardiac condition of the ambulatory patient.

In examples of the ambulatory externally worn cardiac device, the ambulatory exertion test can include one or more of a distance-based walk test, a distance-base run test, a distance-based climb test, a time-base walk test, a time-base run test, and/or a time-based climb test.

In examples of the ambulatory externally worn cardiac device, the one or more motion detectors can include one or more accelerometers configured to generate a plurality of motion signals representative of movement of a patient. In some examples, the at least one processor can be configured to periodically determine the current activity level-dependent parameter of the ambulatory patient by being further configured to analyze the plurality of motion signals to determine if at least a portion of the plurality of motion signals exceed a motion threshold and generate a motion classification based on the determine that at least a portion of the plurality of motion signals exceed the motion threshold, the motion classification including a motion type and a current level of activity of the ambulatory patient. In some examples, the motion threshold can include a change in signal amplitude on at least one directional axis of the plurality of motion signals that exceeds 50% over a period of time, a change in signal on at least one directional axis of the plurality of motion signals that exceeds 100% over a period of time, and/or a change in signal on at least one directional axis of the plurality of motion signals that exceeds 150% over a period of time. In some examples, wherein the period of time can include thirty seconds, one minute, two minutes, five minutes, and/or ten minutes.

In another example, a non-transitory computer-readable medium storing computer-executable instructions to implement a process to automatically assess a physical condition of an ambulatory patient wearing an ambulatory externally worn cardiac device is provided. The instructions include instructions to receive, for the ambulatory patient wearing the device, initiating criterion corresponding to an ambulatory exertion test, wherein the initiating criterion specifies heartrate-dependent parameter requirements and/or activity level-dependent parameter requirements; continuously monitor a current heartrate-dependent parameter and/or a current activity level-dependent parameter of the ambulatory patient; periodically determine whether the monitored current heartrate-dependent parameter and/or the current activity level-dependent parameter satisfies the initiating criterion for the ambulatory exertion test; invite, via a user interface prompt, the ambulatory patient to participate in the ambulatory exertion test; and initiate, upon receiving patient input indicating acceptance of the invitation, the ambulatory exertion test.

Implementations of the non-transitory computer-readable medium storing computer-executable instructions to implement a process to automatically assess a physical condition of an ambulatory patient wearing an ambulatory externally worn cardiac device can include one or more of the following features.

In examples of the non-transitory computer-readable medium, the heartrate-dependent parameter can include one or more of heartrate, energy exerted by the ambulatory patient, work performed by the ambulatory patient, heartrate variability, premature ventricular contraction burden or counts, atrial fibrillation burden, pauses, heartrate turbulence, QRS height, QRS width, changes in ECG morphology, cosine R-T, QT interval, QT variability, T-wave width, T-wave alternans, T-wave amplitude, T-wave variability, R-wave amplitude, and ST segment changes.

In examples of the non-transitory computer-readable medium, the current activity level-dependent parameter can include one or more of activity type, activity level, activity duration, energy exerted by the ambulatory patient, and work performed by the ambulatory patient.

In examples of the non-transitory computer-readable medium, the instructions can further include instructions to receive a prescription for an ambulatory exertion test for the ambulatory patient from a healthcare provided associated with the ambulatory patient, the prescription including exertion test initiating criteria. In some examples, the instructions can further include instructions to periodically determine whether the monitored current heartrate-dependent parameter and/or the current activity level-dependent parameter satisfies the exertion test initiating criteria, invite, via a user interface prompt, the ambulatory patient to participate in the ambulatory exertion test, and initiate, upon receiving patient input indicating acceptance of the invitation, the ambulatory exertion test. In some examples, the ambulatory exertion test can include a physical test directed to the ambulatory patient and is configured to prompt the ambulatory patient to perform a particular activity for a predetermined period of time. In some examples, the physical test can include a walking test, a jogging test, a running test, and/or a stair climbing test. In some examples, the predetermined period of time can include at least one of one minute, two minutes, five minutes, six minutes, ten minutes, fifteen minutes, twenty minutes, and/or thirty minutes.

In examples of the non-transitory computer-readable medium, the initiating criterion can further include monitoring the ambulatory patient performing a motion activity for a minimum period of time as determined for the ambulatory patient. In some examples, the motion activity can include walking, running, and/or climbing stairs. In some examples, the minimum period of time can include thirty seconds, one minute, two minutes, three minutes, five minutes, and/or ten minutes. In some examples, the ambulatory exertion test can include a physical test directed to the ambulatory patient and configured to prompt the ambulatory patient to perform a particular activity for a predetermined period of time. In some examples, the physical test can include a walking test, a jogging test, a running test, and/or a stair climbing test. In some examples, the predetermined period of time can include at least one of one minute, two minutes, five minutes, six minutes, ten minutes, fifteen minutes, twenty minutes, and/or thirty minutes.

In examples of the non-transitory computer-readable medium, the instructions can further include instructions to monitor the ambulatory patient during the ambulatory exertion test, determine if the ambulatory patient is satisfying at least one sustaining criterion during the ambulatory exertion test, the at least one sustaining criterion indicating whether the ambulatory patient is satisfactorily completing the ambulatory exertion test, and terminate the ambulatory exertion test if the ambulatory patient does not satisfy the at least one sustaining criterion. In some examples, the at least one sustaining criterion can include a minimum level of activity to be maintained by the ambulatory patient during the ambulatory exertion test and/or a target heart range for the ambulatory patient during the ambulatory exertion test.

In examples of the non-transitory computer-readable medium, the instructions can further include instructions to monitor the ambulatory patient during the ambulatory exertion test, determine that the ambulatory patient has satisfied at least one terminating criterion for the ambulatory exertion test, provide an indication to the ambulatory patient that the ambulatory patient has completed the ambulatory exertion test, and terminate the ambulatory exertion test. In some examples, the instructions to terminate the ambulatory exertion test can include instructions to generate a report including patient performance information as measured during the ambulatory exertion test. In some examples, the at least one terminating criterion can include a total distance walked, a total distance run, a total number of stairs climbed, and/or a total time elapsed.

In examples of the non-transitory computer-readable medium, the instructions can further include instructions to monitor a sensed cardiac electrical signal and at least one motion signal for the ambulatory patient during the ambulatory exertion test and determine patient performance information for the ambulatory patient during the ambulatory exertion test. In some examples, the instructions can further include instructions to output at least one performance message to the ambulatory patient during the ambulatory exertion test, the performance message including one or more of an indication to increase pace, an indication to decrease pace, an indication to maintain current pace, an indication of time remaining, and/or an indication of elapsed time. In some examples, the instructions can further include instructions to output at least a portion of the performance information to the ambulatory patient during the ambulatory exertion test, the performance information including one or more of total steps taken during the ambulatory exertion test, total distance covered during the ambulatory exertion test, total stairs climbed/descended during the ambulatory exertion test, average heartrate during the ambulatory exertion test, maximum heartrate during the ambulatory exertion test, and/or minimum heartrate during the ambulatory exertion test.

In examples of the non-transitory computer-readable medium, the instructions can further include instructions to monitor cardiac activity for the ambulatory patient during the ambulatory exertion test based upon at least one sensed cardiac electric signal, determine if the ambulatory patient is experiencing a cardiac arrhythmia during the ambulatory exertion test, and terminate the ambulatory exertion test if the ambulatory patient experiences a cardiac arrhythmia.

In examples of the non-transitory computer-readable medium, the instructions can further include instructions to monitor current motion activity for the ambulatory patient during the ambulatory exertion test based upon at least one motion signal, determine if the ambulatory patient is experiencing a fall event during the ambulatory exertion test, and terminate the ambulatory exertion test if the ambulatory patient experiences a fall event.

In examples of the non-transitory computer-readable medium, the instructions can further include instructions to monitor the ambulatory patient during a baseline ambulatory exertion test and determine baseline performance information for the ambulatory patient based upon the baseline ambulatory exertion test. In some examples, the instructions can further include instructions to monitor the ambulatory patient during the ambulatory exertion test, determine if the ambulatory patient is satisfying at least one sustaining criterion during the ambulatory exertion test, the at least one sustaining criterion being based upon the baseline ambulatory exertion test and indicating whether the ambulatory patient is satisfactorily completing the ambulatory exertion test, and terminate the ambulatory exertion test if the ambulatory patient does not satisfy the at least one sustaining criterion. In some examples, the instructions can further include instructions to generate patient performance information for the ambulatory patient upon completion of the ambulatory exertion test, compare the ambulatory patient performance information to the baseline performance information to produce comparison exertion test information, and determine at least one trend for the ambulatory patient based upon the comparison exertion test information, the at least one trend indicating a change in an overall cardiac condition of the ambulatory patient.

In examples of the non-transitory computer-readable medium, the instructions can further include instructions to administer a plurality of ambulatory exertion tests to the ambulatory patient, record patient performance information for each of the plurality of ambulatory exertion tests to generate historical patient performance information, administer a new ambulatory exertion test to the ambulatory patient, record updated patient performance information for the new ambulatory exertion test, compare the updated patient performance information to the historic patient performance information to produce comparison exertion test information, and determine at least one trend for the ambulatory patient based upon the comparison exertion test information, the at least one trend indicating a change in an overall cardiac condition of the ambulatory patient.

In examples of the non-transitory computer-readable medium, the ambulatory exertion test can include one or more of a distance-based walk test, a distance-base run test, a distance-based climb test, a time-base walk test, a time-base run test, and/or a time-based climb test.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples and are incorporated in and constitute a part of this specification but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIGS. 1A-1C illustrate sample motion sensor arrangements for a patient, in accordance with an example of the present disclosure.

FIG. 2 illustrates the output of a sample accelerometer, in accordance with an example of the present disclosure.

FIG. 3 illustrates a schematic view of a sample controller for a wearable medical device, in accordance with an example of the present disclosure.

FIG. 4 depicts a sample network overview, in accordance with an example of the present disclosure.

FIG. 5 depicts a second sample network overview, in accordance with an example of the present disclosure.

FIG. 6 depicts a sample process flow for determining when to initiate an exertion test, in accordance with an example of the present disclosure.

FIG. 7 depicts a sample process flow for receiving testing criteria from a healthcare provider, in accordance with an example of the present disclosure.

FIGS. 8A and 8B illustrate sample user interfaces that a physician can access to generate testing criteria for a cardiac patient, in accordance with examples of the present disclosure.

FIG. 9 depicts a sample process flow for determining whether to sustain or terminate an exertion test, in accordance with an example of the present disclosure.

FIGS. 10A and 10B illustrate sample screenshots of a wearable medical device prior to, during, and after an exertion test, in accordance with examples of the present disclosure.

FIG. 11 depicts a sample process flow for determining health trend information for a patient, in accordance with an example of the present disclosure.

FIG. 12 depicts an alternate sample process flow for determining health trend information for a patient, in accordance with an example of the present disclosure.

FIGS. 13A-13B illustrate sample accelerometer data, in accordance with examples of the present disclosure.

FIGS. 14A-14D illustrate sample ambulatory medical devices that may be prescribed to a heart failure patient, in accordance with an example of the present disclosure.

FIG. 15A illustrates a patient force vectors versus time during a fall event where a patient falls to the ground, in accordance with examples of the present disclosure.

FIG. 15B illustrates a graph of patient motion vectors versus time during fall event where a patient falls to the ground, in accordance with examples of the present disclosure.

### DETAILED DESCRIPTION

Wearable medical devices, such as cardiac event monitoring and treatment devices, are used in clinical or outpatient settings to monitor and/or record various ECG and other physiological signals of a patient. These ECG and other physiological signals can be used to determine a current condition of a patient, monitor for arrhythmias, and provide treatment such as a defibrillating/cardioverting shock in the event of life-threatening arrhythmias.

Additionally, for many heart failure patients, a physical rehabilitation plan, or cardiac rehabilitation plan, is prescribed to improve the physical condition of the patient. Individuals with cardiac disease or who have experienced a cardiac event typically benefit from participation in a cardiac rehabilitation plan that includes regular exercise and lifestyle changes. Cardiac rehabilitation can include a coordinated and multifaceted approach to recovery that is designed to reduce risk, foster and sustain performance of healthy behaviors, reduce disability, and promote an active lifestyle for heart failure patients.

At least some examples described herein are directed to wearable medical devices and methods that guide patients to complete one or more physical exertion tests. Wearable medical devices are continuously worn and so can continuously monitor ECG and other physiological information of ambulatory patients. Additionally, wearable medical devices as described herein incorporate one or more motion sensors configured to monitor motion data for the patient wearing the device. The systems, devices, and techniques herein implement and administer physical exertion tests to patients that draw on such continuously available real-time physiological information and motion information in at least two ways. First, the device is configured to provide and administer physical exertion tests that are dynamically adjusted based on the most current and/or real-time ECG and non-ECG physiological information from the ambulatory patient. Second, the device is configured to measure the patient's performance during a physical exertion test based on the most current and/or real-time ECG and non-ECG physiological information and motion information from the ambulatory patient.

Cardiac rehabilitation resulting from physical exertion tests as described herein can be delivered in both in-patient and outpatient settings and has been shown to reduce the rate of mortality and morbidity in heart failure patients by stabilizing, slowing, or even reversing the progression of certain cardiac diseases. Clinical data demonstrates that engaging in properly administered outpatient cardiac rehabilitation is safe and reduces morbidity, mortality, and readmission rates. Various studies have shown that patients who participate in outpatient cardiac rehabilitation have a lower risk of death (e.g., 21%-47%), a lower risk of myocardial infarction (e.g., 21%-31%), and decreased readmissions as compared to patients who do not participate in outpatient cardiac rehabilitation. The benefits provided by physical exertion testing features, systems, devices, and techniques as described herein are important to the individual heart failure patient and to society as subsequent healthcare costs may be reduced following participation.

Unfortunately, approximately 45%-60% of patients are never referred to an outpatient cardiac rehabilitation program for physical exertion testing and approximately 25% who are referred do not enroll in the program. Moreover, patient adherence to cardiac rehabilitation plans is problematic, with average dropout rates running as high as 50%. There are several reasons given for these discouraging statistics, but a predominate reason listed amongst cardiac patients that do not enroll in outpatient cardiac rehabilitation is the requirement to attend scheduled rehabilitative exercise sessions at a specific facility or office. A patient may be required to attend a number of office visits throughout the rehabilitation period, usually multiple visits a week. For some patients with limited mobility or transportation resources, such a commitment becomes burdensome and the cardiac rehabilitation plan is partially or completely ignored, and the patient's physical recovery directly suffers. Thus, many of the benefits of cardiac rehabilitation described above remain unrealized for many patients.

To address these and other obstacles to successful execution of, and patient adherence to, monitored physical exertion tests, methods and systems are described herein to facilitate remote physical exertion tests that are individualized to each patient and dynamically adjusted. These features increase the convenience of the physical exertion tests and decrease the discomfort felt by the patient by aligning exercise frequency, intensity, and duration with the patient's current medical condition. Thus, the methods and systems described herein provide a patient and their healthcare provider (HCP) with the ability to engage in remote physical exertion tests as outlined in, for example, a cardiac rehabilitation plan while still providing the benefits of dynamically adjusting physical exertion test characteristics such as length of test and testing frequency based upon patient response to the physical exertion tests over time.

As a further benefit of the systems and techniques disclosed herein, patients may carry out their daily activities and be reminded to perform the physical exertion test when the conditions are right (as determined by the patient's individual lifestyle, habits, needs, and schedule). For example, a patient out on a walk may, after a certain duration, be prompted to extend the walk in order to carry out and complete a prescribed physical exertion test.

In certain implementations of the present disclosure, methods and systems use the monitoring and processing capabilities of ambulatory medical devices to monitor patient physiological information and patient movement information as the patient goes about their daily activities. In some implementations, the ambulatory medical device can also track how the patient's body responds to the physical exertion test-before, during, and after its administration. To monitor and protect the patient from life-threatening arrhythmias during the exertion tests, the ambulatory medical device can include arrhythmia treatment elements, for example, as in the case of a wearable cardioverter/defibrillator (WCD) or an externally worn device transcutaneous pacing device.

In certain implementations, the various physiological information and patient motion information collected by the medical device can be used to determine whether and when the patient should be prompted to or self starts a physical exertion test (e.g., a start time), how long the patient performs the physical exertion test (e.g., test time duration), and when the patient ends the physical exertion test (e.g., an end time measured as the test time duration added to the start time). For specific exercises such as walking, the physiological information and motion information can be used to determine additional activity-specific information such as how far the patient travels during the exercise session, what their average step rate is during the exercise session, what their maximum step rate is during the exercise session, whether the patient rests during the exercise session, and other similar information. The physiological information can also be used to determine additional physical response data for the patient such as maximum heartrate during the exercise session, average heartrate during the exercise session, average respiratory rate during the exercise session, maximum respiratory rate during the exercise session, ventricular tachycardia (VT)/ventricular fibrillation (VF) onset occurrence, and other similar physiological metrics.

In some implementations, the methods and systems further utilize the processing capabilities of a remote server and/or the medical device to update testing criteria for a patient as well as to identify health trend information for a patient. This server can include a network interface in secure wireless communication with the wearable medical device and at least one processor configured to execute a variety of processes. For instance, in these implementations, the processor has access to a database housing one or more sets of testing criteria. The processor can execute a exertion test generation process that parses input received from an HCP and generates a set of testing criteria for a particular patient. The test generation process can store the generated testing criteria within the database. In some examples, each set of testing criteria can also be associated with start times, and durations and/or end times to fully customize a physical exertion test for a specific patient.

In some implementations, the server can cause the patient's wearable medical device to administer the physical exertion test based on the one or more plan data structures. For instance, the server can transmit a message via a network connecting the server and the wearable medical device. The message can include data descriptive of the physical exertion test and a request for the wearable medical device to begin administration of the physical exertion test when the patient being tested satisfies an initiating criterion. In response to receipt of the message, the wearable medical device can notify, via a user interface, the patient of details regarding the physical exertion test, provide reminders to perform a physical exertion test, and execute other processes that administrate the physical exertion test. As described above, the wearable medical device can further track the patient's performance during physical exertion tests. In addition, the wearable medical device can transmit messages to the server that communicate the patient's ECG and non-ECG physiological information and additional physical response data as monitored during a physical exertion test.

In certain implementations, the server can receive and process the collected and determined information from the patient's wearable medical device. For example, the server can periodically or continuously parse the messages containing the physiological information and determine any trends related to the patient's current health based upon the information. For example, the server can compare updated patient performance information from a recently completed physical exertion test against historic patient data to determine any changes in the patient's health. These changes can include, for example, changes in average heartrate during the physical exertion test, time to return to normal resting heart after the physical exertion test, changes in fatigue and/or shortness of breath experienced by the patient during and/or after a physical exertion test, and other similar changes.

In some examples, the system (e.g., the server and/or the wearable medical device) can implement a user interface through which the system can receive input from the HCP regarding initiating, sustaining, and terminating criteria for a physical exertion test as described herein. This user interface can include controls that display and/or receive input regarding elements of a physical exertion test. In this way, these example systems enable an HCP to prescribed personalized physical exertion tests for an individual patient.

The methods and systems described herein provide several advantages. For example, one advantage of the examples described herein is that the patient need not attend regular HCP clinical office visits during which physical exertion tests are administered. Rather, the wearable medical device automatically monitor's the patient's performance during physical exertion tests. Additionally, the examples disclosed herein enable a wide population of ambulatory cardiac rehabilitation patients to participate, following a physician's or other HCP's referral, in physical exertion testing that focuses on preventative and rehabilitative services. This population of patients can include hospitalized patients, patients recently discharged from hospital following a cardiac event or procedure, and/or out-patients who have recently been diagnosed with being at risk of a cardiac event. In some implementations, the methods and systems described herein administer physical exertion tests that focus on assessment of current clinical condition, patient mobilization, and identification and provision of information regarding modifiable risk factors and self-care.

These examples, and various other similar examples of benefits and advantages of the techniques, processes, and approaches as provided herein, are described in additional detail below.

The various monitoring processes as described herein are implemented, in some examples, by data processing devices, such as computer systems and certain types of medical devices. For instance, some examples include a patient monitoring and treatment device. Patient monitoring and treatment devices are used to monitor and record various physiological or vital signals for a patient and provide treatment to a patient when necessary. For patients at risk of a cardiac arrhythmia, specialized cardiac monitoring and/or treatment devices such as a cardiac event monitoring device, a WCD, or a hospital wearable defibrillator can be prescribed to and worn by the patient for an extended period of time. For example, a patient having an elevated risk of sudden cardiac death, unexplained syncope, prior symptoms of heart failure, an ejection fraction of less than 45%, less than 35%, or other such threshold deemed of concern by a physician, and other similar patients in a state of degraded cardiac health can be prescribed a specialized cardiac monitoring and/or treatment device.

For example, a WCD such as the LifeVest® Wearable Cardioverter Defibrillator from ZOLL Medical Corporation (Chelmsford, MA), can be prescribed to the patient. As described in further detail below, such a device includes a garment that is configured to be worn about the torso of the patient. The garment can be configured to house various components such as ECG sensing electrodes, therapy electrodes, and one or more accelerometers configured to measure motion data for the patient. The components in the garment can be operably connected to a monitoring device that is configured to receive and process signals from the ECG sensing electrodes to determine a patient's cardiac condition and, if necessary, provide treatment to the patient using the therapy electrodes.

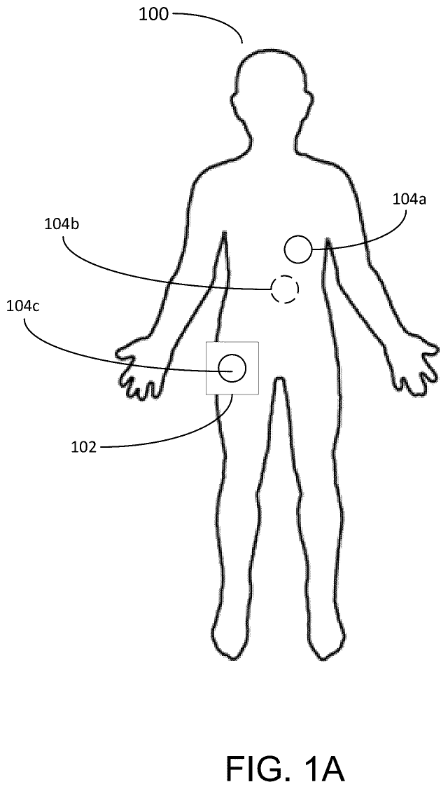

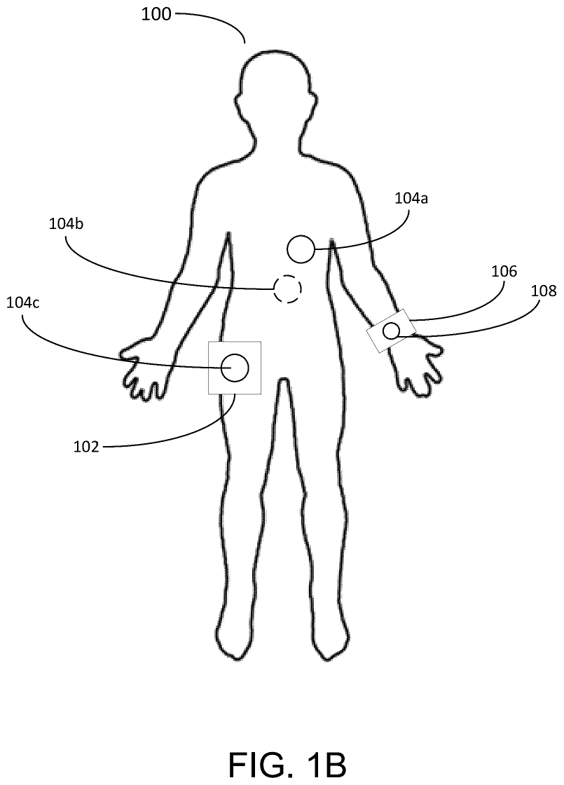

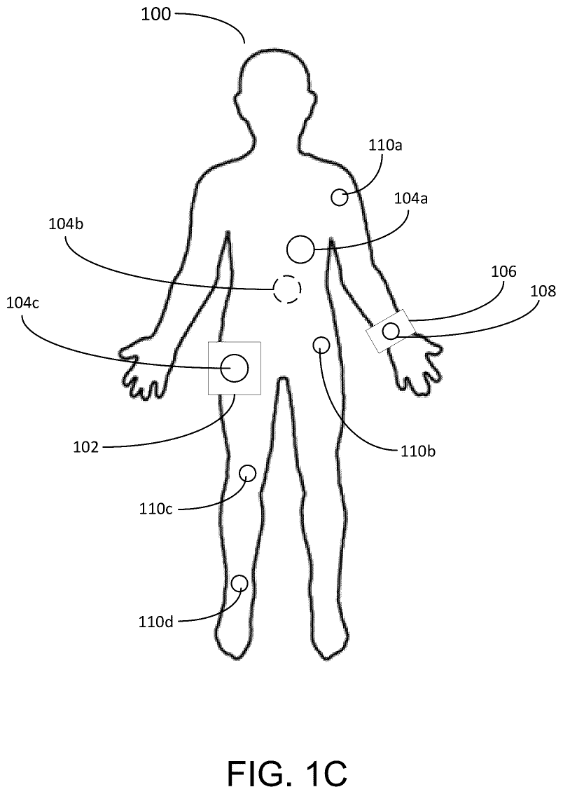

FIGS. 1A-1C illustrate various examples of a patient 100 wearing one or more motion sensors such as accelerometers as described herein. It should be noted that accelerometers are described herein as examples of motion sensors for illustrative purposes only. In certain implementations, additional motion sensors such as gyroscopes, magnetic sensors, pressure-based motion sensors, and other similar motion sensors can be used.

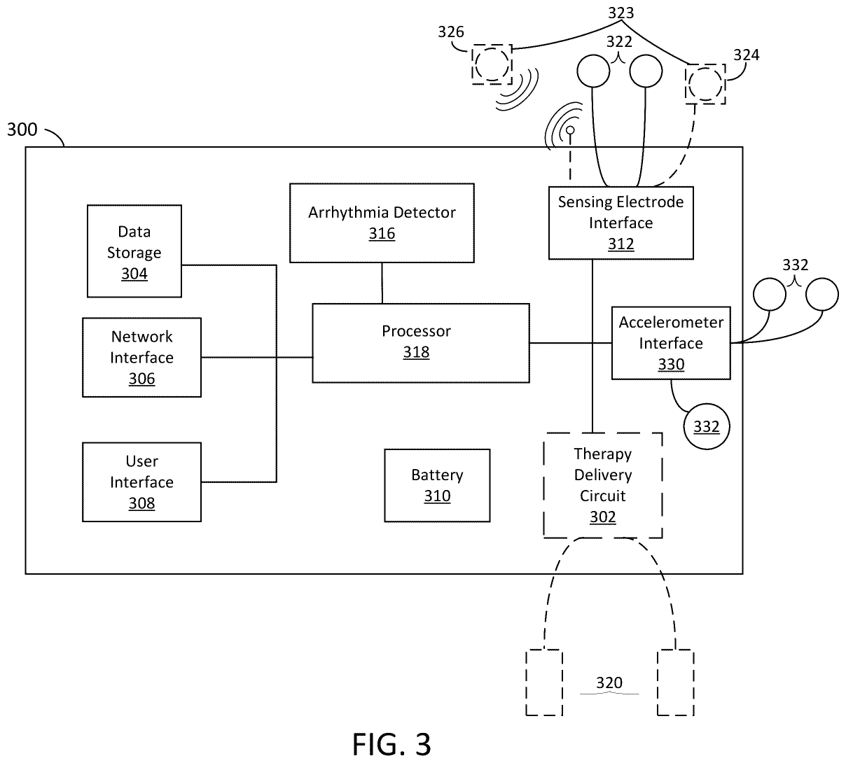

As shown in FIG. 1A, a patient can be prescribed an ambulatory medical device such as a WCD. The WCD can include a controller 102 that is operably connected to one or more sensing electrodes and therapy electrodes. Additional details of one example of the controller 102 can be found in the discussion of FIG. 3 below.

The WCD can also include one or more accelerometers or other motion sensors. As shown in FIG. 1A, the WCD can include three accelerometers 104a, 104b, and 104c (collectively referred to as accelerometers 104) positioned at various places on the body of patient 100. For example, accelerometer 104a can be positioned on the front of chest of the patient 100, the accelerometer 104b can be positioned on the back of the patient, and the accelerometer 104c can be integrated into the controller 102. Each of the accelerometers 104 can be configured to measure movement associated with the patient 100 and to output an electrical signal indicating a direction and magnitude of the movement of the patient.

It should be noted that the number and arrangement of the accelerometers 104 as shown in FIG. 1 is by way of example only. In certain implementations, the number and position of the accelerometers 104 can vary. Additionally, when included in a device such as a WCD, one or more of the accelerometers 104 can be integrated into components of the WCD. For example, as noted above, the accelerometer 104c can be integrated into a controller 102 of the WCD. Similarly, one or more of accelerometers 104a and 104b can be integrated into one or more components of a WCD. For example, the front accelerometer 104a can be integrated into, for example, a therapy electrode operably connected to the controller 102 and configured to provide a therapeutic shock to the patient 100. In some implementations, the accelerometer 104a can be integrated into a sensing electrode configured to measure electrical signals produced by the patient 100 and indicative of cardiac activity of the patient 100. Similarly, accelerometer 104b can be integrated into one or more components of a WCD such as a connection node, a sensing electrode, a therapy electrode, and other similar components of a WCD as described herein.

In addition to accelerometers associated with a WCD as described above in regard to FIG. 1A, a patient such as the patient 100 can also wear additional accelerometers integrated into, for example a wearable motion detection device such as a fitness tracker. As shown in FIG. 1B, the patient 100 can wear a wrist-worn motion tracking device 106. For example, the device 106 can be implemented as a fitness tracker configured to measure movement and translate the movement into indications of physical activities such as walking, running, climbing stairs, and other similar physical activity. In some examples, the device 106 can be implemented as a standalone fitness tracking device or be implemented into another device such as a smartphone and/or smartwatch.

As further shown in FIG. 1B, the device 106 can include one or more additional accelerometers 108. In the example shown in FIG. 1B, the accelerometers 104 can be on the trunk or core of the patient 100 and can be configured to measure overall body movements while accelerometers 108 can be positioned on the wrist (or another similar extremity) and be configured to measure motion of the arm (or other extremity) of the patient.

However, it should be noted that device 106 and accelerometers 108 are shown by way of example only. In some implementations, a patient such as the patient 100 can wear additional accelerometers that are configured to collect additional motion data for the patient. For example, as shown in FIG. 1C, the patient 100 can wear additional accelerometers located on different portions of the patient's body such as left or right shoulder, 110a, left or right upper leg or left or right hip area 110b, left or right mid-leg or knee area 110c, and left or right lower leg 110d (collectively referred to as accelerometers 110). In some examples, the accelerometers 110 can be integrated into one or more articles of clothing or wearable garments such as a belt configured to go around the waist or chest of the patient 100, a brace or strap configured to go around the knee or ankle of the patient, and other similar articles of clothing or wearable garments. In examples, one or more of the accelerometers 110 can be wireless accelerometers. For example, a wireless accelerometer can include a tilt sensor such as part number MNS-9-W1-AC-TL from Monnit of Salt Lake City, Utah, USA.

It should be noted that the placement and number of accelerometers as shown in FIGS. 1A-1C are shown by way of example only. In actual implementation of the exertion testing processes as described herein, the number and position of the accelerometers can vary based upon the anticipated activity level of the patient, the type of activity the patient is expected to undertake, and other various factors.

To properly acquire and output a signal indicative of a patient's movement, an accelerometer such as those described above in FIGS. 1A-1C can be configured to output one or more output signals indicative of any detected movement or motion. As described herein, patient motion information can be monitored and recorded during an administered exertion test for later analysis and review. Patient motion information can be constructed from motion data as output by the accelerometers as described herein. In some examples, the motion data can be analyzed in a sliding time window between, for example, two and twenty seconds. The analysis can generate outputs in shorter time periods than the window duration, for example one second outputs, such that the windows overlap in time when constructing the patient motion information. By constructing the motion data into longer strings of information, a processing device can accurately determine what type of activity a patient is performing, how long they have been performing the activity, and can correlate physiological metrics (and changes in those metrics) to the motion information.

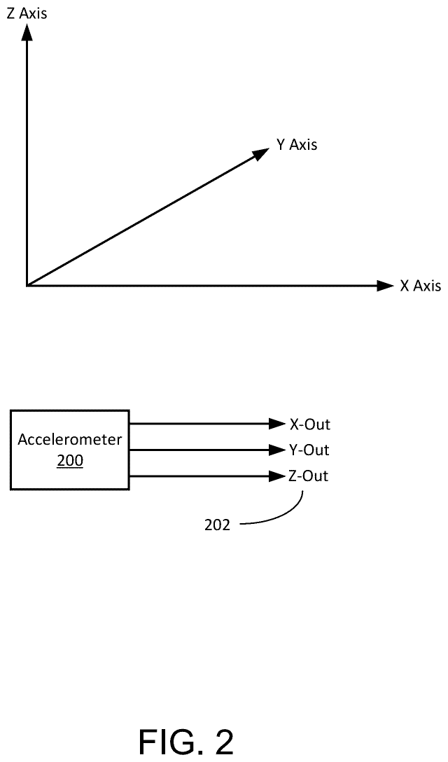

As shown in FIG. 2, an accelerometer 200 can be configured to measure movement in three axes: the x-axis, the y-axis, and the z-axis. Depending upon the orientation of the accelerometer 200 and the output configuration of the accelerometer, the individual axes can define movement of the accelerometer in a specific direction.

Additionally, as shown in FIG. 2, the accelerometer 200 can be configured to provide one or more outputs 202. In this example, the outputs 202 can include an X-out (i.e., a signal indicative of measured movement along the x-axis), a Y-out (i.e., a signal indicative of measured movement along the y-axis), and a Z-out (i.e., a signal indicative of measured movement along the z-axis).

In some implementations, an accelerometer such as accelerometer 200 can be configured to output an electrical signal on each output 202 having one or more controlled characteristics such as voltage. For example, the accelerometer 200 can be configured to output a signal on each output 202 between 0 and 5 volts. In some examples, the output voltage on each output 202 can be directly proportional to measured motion on the corresponding axis. For example, if the accelerometer 200 is configured to measure movement of acceleration as a measure of gravitational forces, the accelerometer can be configured to measure a specific range of g-forces such as −5 g to +5 g. In such an example, the output voltage on each output 202 can be directly proportional to the measured g-force on each axis. For example, of no g-forces are measured (i.e., the accelerometer 200 is at rest), each output signal 202 can be measured at 2.5 volts. If a movement having a positive g-force along an axis is measured, the voltage on the corresponding output 202 can increase. Conversely, if a movement having a negative g-force along an axis is measure, the voltage on the corresponding output 202 can decrease. Table 1 below shows sample voltage output levels for an accelerometer configured to measure between −5 g and +5 g and output a signal between 0 and 5 volts.

TABLE 1

| | |
|---|---|
| Measured G-Force | Output Voltage |

| | | | |
|---|---|---|---|
| −5 | g | 0 | volts |
| −4 | g | 0.5 | volts |
| −3 | g | 1.0 | volts |
| −2 | g | 1.5 | volts |
| −1 | g | 2.0 | volts |
| 0 | g | 2.5 | volts |
| 1 | g | 3.0 | volts |
| 2 | g | 3.5 | volts |
| 3 | g | 4.0 | volts |
| 4 | g | 4.5 | volts |
| 5 | g | 5.0 | volts |

It should be noted that sample g-force and voltage ranges as described above and shown in Table 1 are provided by way of example only for illustrative purposes. Depending upon the design and capabilities of the accelerometers used, the g-force ranges measured, and the corresponding output voltages can vary accordingly.

In certain implementations, raw data from an accelerometer can take the form of a time series of acceleration values in each of the x-axis, the y-axis, and the z-axis. As noted above, raw output from analog accelerometers can be a continuous voltage that is proportional to the acceleration (as shown in Table 1 above) or a square-wave where the duty cycle is proportional to the acceleration (e.g., pulse-width modulation). When using an analog accelerometer, additional circuitry such as an accelerometer interface as described below can be included to provide the acceleration data in a time series.

In certain implementations, the output for each time window for a set of motion data can be represented as a vector:

a=[aₓ,a_{y},a_{z}]

where aₓ, a_{y}, and a_{z} are the x-axis, y-axis, and z-axis components of acceleration as measured by the accelerometer. A time series of accelerometer magnitudes can thus be denoted as:

[∥a[t−NT]∥, . . . ,∥a[t−2T]∥,∥a[t−T]∥,∥a[t]∥

where ∥a[t]∥ is the magnitude of the acceleration vector at time t, T is the sampling period (e.g., 20 milliseconds), and N is the number of consecutive prior samples being analyzed.

In some examples, the output of one or more accelerometers as described herein can be used to determine an activity for a patient if the output exceeds a particular threshold change. For example, a motion threshold for determining patient activity can a change in signal amplitude on at least one directional axis of the plurality of motion signals as output by the accelerometers that exceeds 50% over a period of time, a change in signal on at least one directional axis of the plurality of motion signals that exceeds 100% over a period of time, and/or a change in signal on at least one directional axis of the plurality of motion signals that exceeds 150% over a period of time. In some examples, the period of time can include thirty seconds, one minute, two minutes, five minutes, ten minutes, and other similar periods of time.

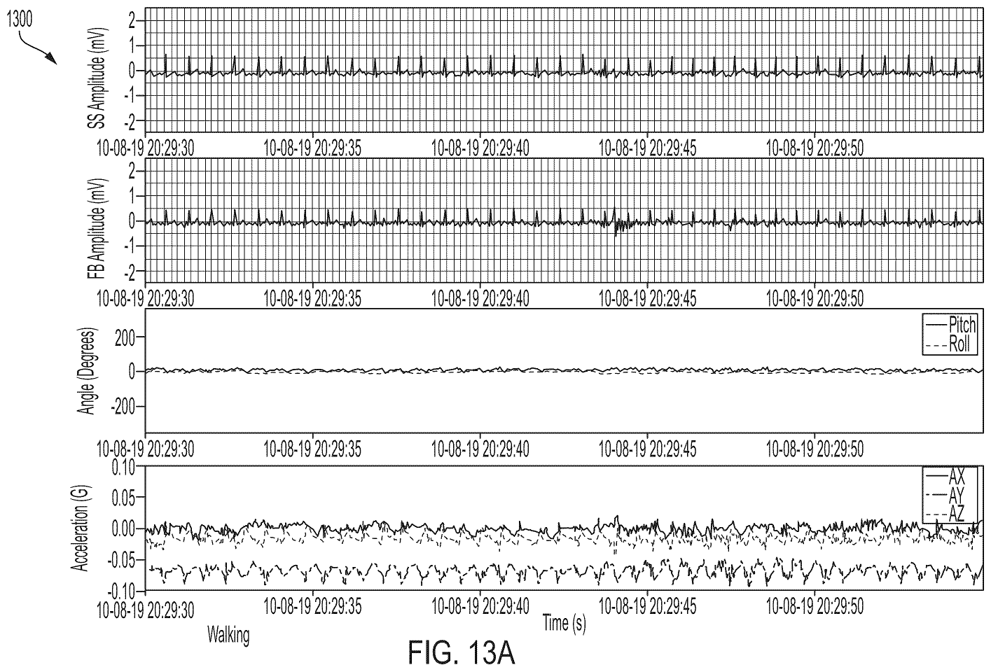

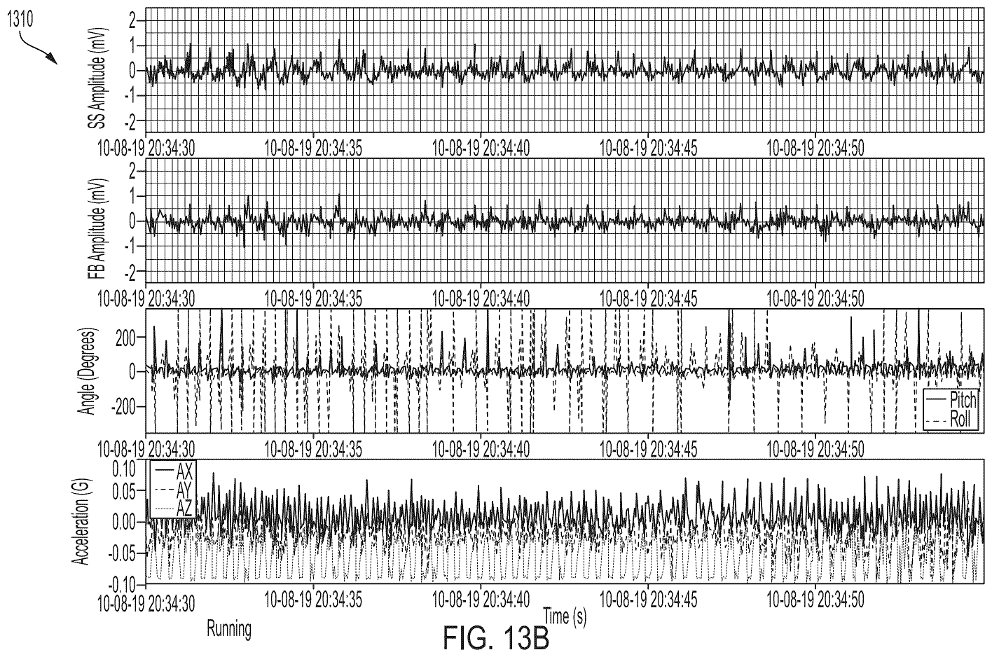

FIGS. 13A and 13B illustrate sample data as output by an accelerometer such as accelerometer 200 as described herein. As shown in FIG. 13A, accelerometer data 1300 shows sample output data as the test subject is walking. For example, as time progresses, the pitch and roll outputs slightly transition above and below 0 degrees. As further shown, the output representing the x-axis transitions slightly above and below about 0 g, the output representing the y-axis transitions slightly above and below about −0.075 g, and the output representing the z-axis transitions slightly above and below about −0.025 g. Using a trained motion classifier model such as those described in, for example, PCT Application No. PCT/US2021/023248 filed Mar. 19, 2021 and titled “Systems and Methods for Classifying Motion of a Patient Wearing an Ambulatory Medical Device,” the content of which is hereby incorporated by reference in its entirety, the output of the accelerometer as shown in FIG. 13A can be classified as walking. For example, TABLE 2 illustrates a set of sample data as output by the motion classifier model indicating that the subject was walking. As shown in TABLE 2, the cell having a (row, column) value of (walking, walking) has a value of 1.0, indicating that the output of the motion classifier model is indicating that the subject is walking.

TABLE 2

| | Laying | | | | | Climbing |
|---|---|---|---|---|---|---|
| | Down | Sitting | Standing | Walking | Running | Stairs |
| Laying | 0.0 | 0.0 | 0.0 | 0.0 | 0.0 | 0.0 |
| Down | | | | | | |
| Sitting | 0.0 | 0.0 | 0.0 | 0.0 | 0.0 | 0.0 |
| Standing | 0.05 | 0.0 | 0.0 | 0.1 | 0.05 | 0.05 |
| Walking | 0.0 | 0.0 | 0.0 | 1.0 | 0.2 | 0.1 |
| Running | 0.0 | 0.0 | 0.0 | 0.1 | 0.2 | 0.1 |
| Climbing | 0.0 | 0.0 | 0.0 | 0.1 | 0.1 | 0.2 |
| Stairs | | | | | | |

As shown in FIG. 13B, accelerometer data 1310 shows sample output data as the test subject is running. For example, as time progresses, the pitch and roll outputs transition quickly above and below zero degrees, sometimes exceeding plus and minus 200 degrees from zero. As further shown, the output representing the x-axis, the y-axis, and the z-axis are transitioning quickly between about 0.1 g and −0.1 g. Using a motion classifier model, the output of the accelerometer as shown in FIG. 13B can be classified as running. For example, TABLE 3 illustrates a set of sample data as output by the motion classifier model indicating that the subject was running. As shown in TABLE 3, the cell having a (row, column) value of (running, running) has a value of 0.95, indicating that the output of the motion classifier model is indicating that the subject is running.

TABLE 3

| | Laying | | | | | Climbing |
|---|---|---|---|---|---|---|
| | Down | Sitting | Standing | Walking | Running | Stairs |
| Laying | 0.0 | 0.0 | 0.0 | 0.0 | 0.0 | 0.0 |
| Down | | | | | | |
| Sitting | 0.0 | 0.0 | 0.0 | 0.0 | 0.0 | 0.0 |
| Standing | 0.05 | 0.0 | 0.0 | 0.0 | 0.1 | 0.05 |
| Walking | 0.0 | 0.0 | 0.0 | 0.1 | 0.2 | 0.0 |
| Running | 0.0 | 0.0 | 0.0 | 0.1 | 0.95 | 0.1 |
| Climbing | 0.0 | 0.0 | 0.0 | 0.0 | 0.1 | 0.1 |
| Stairs | | | | | | |

FIG. 3 illustrates an example component-level view of the medical device controller 300 included in, for example, a wearable medical device such as a WCD. The medical device controller 300 is one example of the controller 102 shown in FIGS. 1A-1C and described above. As shown in FIG. 3, the medical device controller 300 can include a housing 301 configured to house a therapy delivery circuitry 302 configured to provide one or more therapeutic shocks to the patient via at least two therapy electrodes 320, a data storage 304, a network interface 306, a user interface 308, at least one rechargeable battery 310 (e.g., within a battery chamber configured for such purpose), a sensor interface 312 (e.g., to interface with both ECG sensing electrodes 322 and non-ECG physiological sensors 323 such as vibrational sensors, lung fluid sensors, infrared and near-infrared-based pulse oxygen sensor, blood pressure sensors, among others), a cardiac event detector 316, and least one processor 318.

In some examples, the patient monitoring medical device can include a medical device controller 300 that includes like components as those described above but does not include the therapy delivery circuitry 302 and the therapy electrodes 320 (shown in dotted lines). That is, in certain implementations, the medical device can include only ECG monitoring components and not provide therapy to the patient. In such implementations, the construction of the patient monitoring medical device is similar in many respects to a WCD medical device controller 300 but need not include the therapy delivery circuitry 302 and associated therapy electrodes 320.

As further shown in FIG. 3, the controller 300 can further include an accelerometer interface 330 and a set of accelerometers 332. The accelerometer interface 330 can be operably coupled to each of the accelerometers 332 and configured to receive one or more outputs from the accelerometers. The accelerometer interface 330 can be further configured to condition the output signals by, for example, converting analog accelerometer signals to digital signals (if using an analog accelerometer), filtering the output signals, combining the output signals into a combined directional signal (e.g., combining each x-axis signal into a composite x-axis signal, combining each y-axis signal into a composite y-axis signal, and combining each z-axis signal into a composite z-axis signal). In some examples, the accelerometer interface 330 can be configured to filter the signals using a high-pass or band-pass filter to isolate the acceleration of the patient due to movement from the component of the acceleration due to gravity.

Additionally, the accelerometer interface 330 can configure the output for further processing. For example, the accelerometer interface 330 can be configured to arrange the output of an individual accelerometer 332 as a vector expressing the acceleration components of the x-axis, the y-axis, and the z-axis as received from each accelerometer. The accelerometer interface 330 can be operably coupled to the processor 318 and configured to transfer the output signals from the accelerometers 332 to the processor for further processing and analysis.

As described above, one or more of the accelerometers 332 can be integrated into one or more components of a medical device. For example, as shown in FIG. 3, an accelerometer 332 can be integrated into the controller 300. In some examples, an accelerometer 332 can be integrated into one or more of a therapy electrode 320, a sensing electrode 322, a physiological sensor 323, and into other components of a medical device.

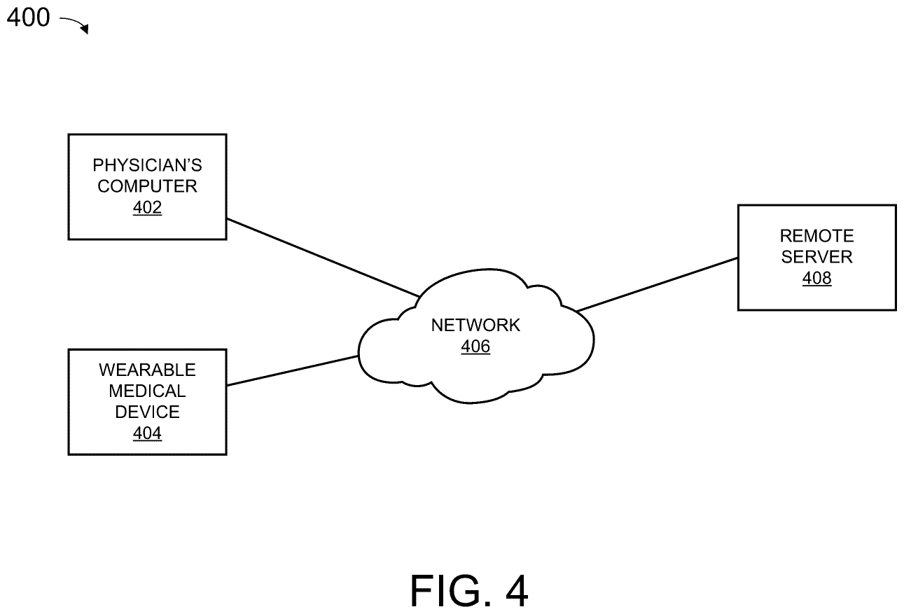

FIG. 4 illustrates a sample network 400 in which a wearable medical device (e.g., a medical device including medical device controller 300 as shown in FIG. 3) can be operably connected to a remote server. As described above in connection with FIG. 3, the wearable medical device controller 300 can include a network interface 306 for transmitting data over a wireless link such as a Bluetooth® wireless link (e.g., via a “hotspot” or other base station or intermediate device), a broadband cellular link, or a Wi-Fi™ communications link based on the IEEE 802.11 standard. As shown in FIG. 4, a doctor's computer 402 and a wearable medical device 404 can be operably connected to a remote server 408 through network 406. In certain implementations, while being worn, the wearable medical device 404 can collect information related to the patient such as various patient metrics and parameters as described herein. For example, the wearable medical device 404 can collect physiological and ECG data related to the patient for the period of time that the patient has been prescribed the wearable medical device. The wearable medical device 404 can also collect accelerometer data indicative of motion information associated with the patient such as number of steps taken during a time period such as 30 minutes, one hour, two hours, six hours, or one day, as well as duration information related to periods of activity such as how often and how long did a patient exercised. Depending upon the connection to network 406, and the programming of wearable medical device 404, the wearable medical device can be configured to regularly transmit the collected information to the remote server 408 for storage and further processing and analysis. The network 406 may be a private, secure network configured to allow for intercommunication between authorized devices 404, computer 402, and server 408. For example, the private network may be implemented as a virtual private network (VPN) that can be formed using a plurality of encrypted communication techniques, including the creation of secure communication channels (called “tunnels”) within a public network. As an example, a device 404 or computer 402 may implement a temporary or permanent dedicated communication software application to securely communicate with the server 408 via such a tunnel. The dedicated communication software application can encrypt and send and messages to the server 408 and receive and decrypt messages received from the server 408. Some types of such dedicated communication software applications can embed encrypted messages in formatted data packets, so that the encrypted messages are unreadable from outside of the secure communication channel.

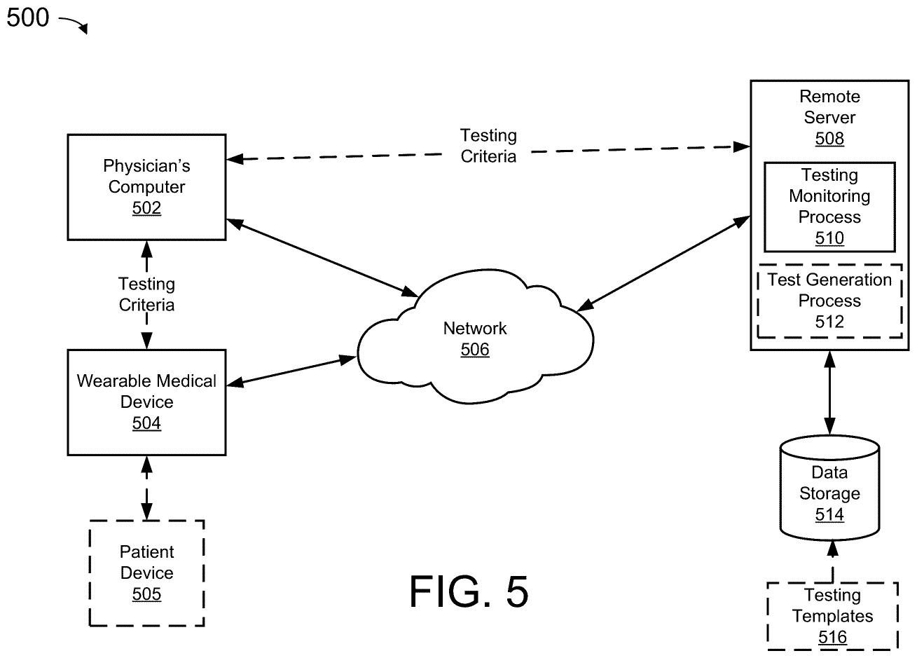

FIG. 5 illustrates a sample network 500 in which a remote server can be operably connected to, and configured to collect information from, a wearable medical device and a physician's computing device. Similar to FIG. 4, a physician's computer 502 and a wearable medical device 504 can be operably connected to a remote server 508 through network 506.

However, to implement the processes and techniques as described herein, the remote server can include a exertion testing monitoring process 510. The monitoring process 510 can be configured to use information collected from the wearable medical device 504 to monitor the patient's results for one or more exertion tests as described herein and to generate one or more reports and/or notifications for review by the patient's healthcare provider based upon the patient's results.

In some examples, the various components as included in network 500 can be configured to exchange information. For example, the doctor's computer 502 can be configured to transfer patient information, exertion testing criteria, alert criteria, patient parameters and/or ECG metrics of interest during monitoring, and other related information to the remote server 508 via the network 506. Similarly, the wearable medical device 504 can be configured to transfer data collected by the wearable medical device including, but not limited to, (a) ECG data, (b) non-ECG data, including motion data, acoustical data, body fluid content information (measured, for example, using radio frequency fluid measurement techniques), and (c) other communication data such as device alerts, indications of device, system and/or arrhythmia conditions, and other similar information to the remote server 508 via the network 506. In certain implementations, the information received from the wearable medical device can include continuously collected information related to the patient. For example, the ECG data can include continuously collected ECG data from which a range of continuous ECG metrics can be derived as described herein. The patient information as collected herein provides for a complete collection of historical data that has been continuously collected and can be further analyzed for any specific time period, e.g., the last 24 hours, last week, two weeks ago, and other similar predefined historical periods as described herein. In some implementations, the ECG data can include discretely recorded ECG strips of predetermined lengths, ranging from a few seconds to several minutes. For instance, the ECG data can include ECG strips of 30 seconds, 1 minute, 2 minutes, 6 minutes, or 10 minutes.

For example, the ECG data can be recorded when a patient experiences and chooses to report a symptom such as fatigue, chest pain, tightness in the chest, a racing heartbeat, dizziness, and/or syncope. In implementations, the ECG data can be automatically recorded when a symptom is detected by a processor integrated into the wearable medical device (e.g., processor 318 as shown in FIG. 3) based on non-ECG physiological data, such as motion data from one or more sensors such as motion sensors. Such symptoms can include changes in a patient's gait, swaying, and a patient fall. For example, the wearable medical device processor can detect one or more such symptoms by establishing a baseline of the patient's normal movements over a period of time. Then, when the patient's gait changes from the established baseline in a manner than transgresses a predetermined threshold, the wearable medical device processor can classify the movement as a change in gait and/or abnormal swaying. When such an event is detected, the patient's ECG data is automatically recorded and stored in a data storage (e.g., data storage 304 as shown in FIG. 3).

In some examples, the wearable medical device can be operably coupled to another patient device 505 such as a patient's computing device. The patient device 505 can be configured to receive data from the wearable medical device 504. In certain implementations, the patient device 505 can include a personal computer, a tablet computer, a smartphone, and other similar computing devices. In some examples, the patient device 505 can be associated with an HCP who is caring for the patient wearing the wearable medical device 504.

The remote server 508 can be operably connected to a data storage device 514 that is configured to store the information received from the doctor's computer 502 and the wearable medical device 504. In some examples, the data storage device 514 can be integrated into the remote server 508 or can be integrated into a separate computing device operably coupled to the remote server.

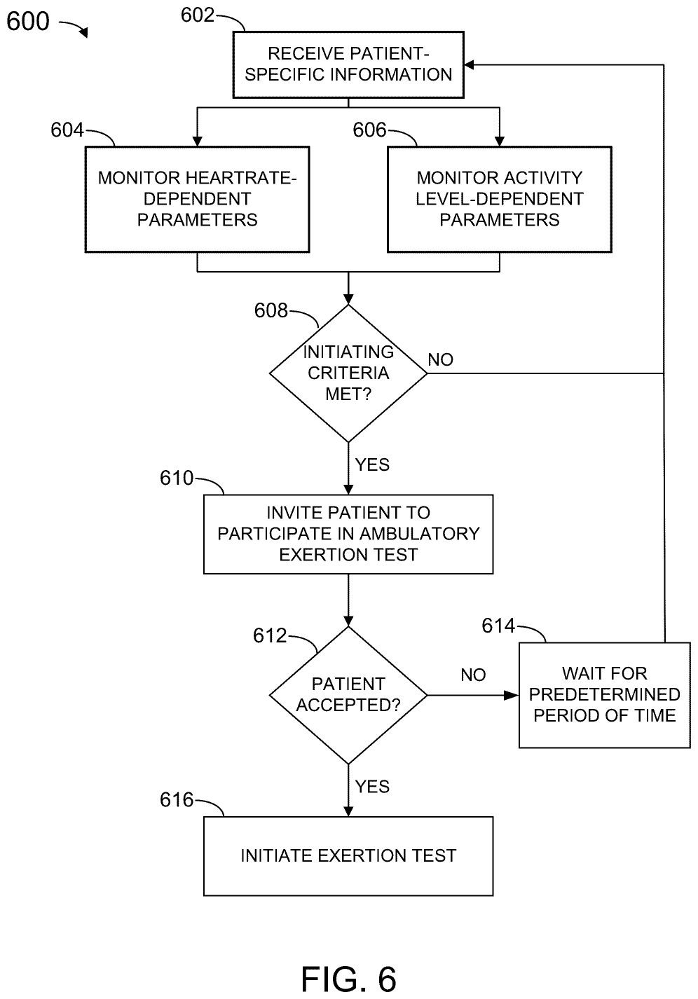

In some examples, the remote server 508 can also include an exertion test generation process 512. The test generation process 512 can be configured, for example, to use patient-specific information and/or testing criteria as received from the physician's computer 502 to generate a patient specific testing criteria for initiating, continuing, and ending an exertion test as described herein. Examples of exertion testing processes, including testing criteria generation processes, are described below in greater detail at, for example, the descriptions of FIGS. 6-8B. In some examples, as shown in FIG. 5, testing criteria can be transmitted directly from the physician's computer 502 to the wearable medical device 504 if, for example, the devices are directly connected (e.g., when the patient is at the physician's office for a routine check-up). It should be noted that the testing criteria being transmitted from the physician's computer 502 to the wearable medical device 504 is shown by way of example only. In certain implementations, the wearable medical device 504 can be configured to receive the testing criteria directly from the remote server 508 such that additional review/analysis of the testing criteria can be performed prior to updating the wearable medical device.

In some examples, the plan generation process 512 as implemented by the remote server 508 can access one or more testing templates 516 stored on the data storage 514 to provide a framework for generating patient-specific testing criteria as described herein. For instance, in certain implementations, the one or more test templates 516 can include one or more pre-set exertion tests that are based upon the current health and expected performance of a cardiac rehabilitation patient. These pre-set exertion tests can be classified, for example, as “easy,” “medium,” and “hard.” Further, in some examples, each of the pre-set cardiac exertion tests can correspond to a classification of a patient, such as provided by the ACC/AHA cardiac patient classification scheme. Thus, in at least one example, the pre-set exertion tests include tests for each of the following stages.
Stage A: Patients at risk for heart failure who have not yet developed structural heart changes (i.e. those with diabetes, those with coronary disease without prior infarct)
Stage B: Patients with structural heart disease (i.e. reduced ejection fraction, left ventricular hypertrophy, chamber enlargement) who have not yet developed symptoms of heart failure
Stage C: Patients who have developed clinical heart failure
Stage D: Patients with refractory heart failure requiring advanced intervention (i.e. biventricular pacemakers, left ventricular assist device, transplantation)

Additionally, in certain implementations the test generation process 512 as implemented by the remote server 508 can access one or more exercise sessions from a library of pre-defined exercise sessions that can be used when determining testing criteria for an individual exertion test.

As described herein, the processor of a wearable medical device, such as processor 318 as shown in FIG. 3 and described herein, can be configured to monitor a patient and, upon the patient satisfying one or more initiating criteria, initiate an exertion test for the patient and monitor the patient's progress through the exertion test. As described herein, a physical exertion test can include instructing or otherwise prompting the patient to perform a particular physical activity for a predetermined period of time. For example, the physical exertion test can include a walking test, a jogging test, a running test, a cycling test, a stair climbing test, and tests including other similar physical activities. The predetermined time period for the physical exertion test can include at least one of one minute, two minutes, five minutes, six minutes, ten minutes, fifteen minutes, twenty minutes, thirty minutes, and other similar time periods. In some examples, the physical exertion test can also be distance based. For example, the physical exertion test can be a w distance-based walking test, a distance-based jogging test, a distance-based running test, a distance-based climbing test, and other similar distance-based physical exertion tests.

Depending upon the patient's physical ability, a physical exertion test as described herein can be initiating upon detecting that the patient is performing a particular physical activity as the patient goes about their day. For example, if patient wearing a medical device such as those described herein is currently walking for an extended period of time (e.g., more than 2 minutes), the device can prompt the patient to participate in a physical exertion test that includes a time or distance-based walking activity. Such a prompt can require the patient to extend their walk or, in some instances, the test may simply be a portion of the walk that the patient was already taking and does not interfere with or otherwise change their daily routine.

In some examples, the patient performing a similar activity to that as included in one or more physical exertion tests as described herein can prompt the medical device to invite the patient to complete a physical exertion test. For example, if the patient is cleaning their house, the medical device can detect that the patient is both walking and climbing stairs. In response to such a detection, the medical device can prompt the patient to perform a physical activity test associated with walking and/or climbing stairs.

In certain implementations, the medical device can be configured to monitor for patterns in the physical activity of the patient and schedule/initiate physical exertion tests accordingly. For example, if the patient regularly goes for a walk at 8:00 am for a particular number of days (e.g., for 3 or more days a week), the medical device can be configured to prompt the patient during those walks to perform a physical exertion test that includes a walking activity. In some examples, the patient can access a portal or other similar interface on the medical device (or through a connected device such as patient device 505 as shown in FIG. 5 and described above) to provide activity information. For example, if the patient regularly visits a gym to exercise, the patient can provide that information through the patient portal. Upon receiving this information, the medical device can update its testing schedule such that prompts for a physical exertion test are more likely to be delivered to the patient during times that the patient has indicated they are likely to be exercising.

FIG. 6 illustrates process 600 that includes determining whether a physical exertion test as described herein is to be initiated for a particular patient. As shown in FIG. 6, process 600 can include the processor receiving 602 patient-specific information. In certain implementations, the patient-specific information can include heart-rate information and motion information for the patient as described herein.

The processor can be configured to monitor 604 the heartrate-dependent parameters as described herein. For example, the heartrate-dependent parameters can include one or more of heartrate, energy exerted by the ambulatory patient, work performed by the ambulatory patient, heartrate variability, premature ventricular contraction burden or counts, atrial fibrillation burden, pauses, heartrate turbulence, QRS height, QRS width, changes in ECG morphology, cosine R-T, QT interval, QT variability, T-wave width, T-wave alternans, T-wave amplitude, T-wave variability, R-wave amplitude, and ST segment changes. Similarly, the processor can be configured to monitor 606 the activity level-dependent parameters. For example, based upon the output of the one or more accelerometers as described herein, the processor can determine and monitor one or more activity level-dependent parameters. In certain implementations, the one or more activity level-dependent parameters can include one or more of activity type, activity level, activity duration, energy exerted by the ambulatory patient, and work performed by the ambulatory patient.

As described herein, the processor can implement process 600 as the patient goes about their day. As such, monitoring 606 the patient's activity level-dependent parameters can also include determining what current physical activity the patient is performing. For example, if the patient is walking, the processor can monitor 606 information related to the patient walking such as duration of walking, total distance traveled, steps per minute, and other similar activity information. Additionally, as noted above, the patient can provide personal exercise schedule information to the medical device (or, in some examples, the medical device can monitor for and determine times that the patient is likely to be exercising). In such an example, the processor can also monitor for times when the patient is likely to be exercising based upon historic exercise and/or daily activity information.

As further shown in FIG. 6, the processor can determine 608 whether one or more initiating criteria have been met by the patient. For example, the initiating criteria can include both heartrate-dependent criteria as well as activity level-dependent criteria. In certain implementations, the heartrate-dependent criteria can include heartrate measurement within a particular threshold range or, in some examples, below a particular threshold. For example, a heartrate-dependent criterion can define that a patient's heartrate must be between 60 bpm and 100 bpm to initiate a test. Similarly, the activity level-dependent criteria can include determining that the patient is already performing a particular activity as the patient goes about their normal daily routine. For example, the processor can determine that the patient is already walking. Such a determination can satisfy an activity level-dependent criterion for initiating a physical exertion test that includes walking.

In some examples, the initiating criterion can include determining that the patient has been performing a physical activity for a minimum period of time. For example, if the patient has been walking for at least five minutes, the processor can determine 608 that the patient has satisfied the initiating criteria for administering a walking-based physical exertion test. In some examples, the monitored motion activity of the patient can include running, jogging, climbing stairs, and other similar physical activities. In some examples, the minimum amount of time that the patient has been performing the physical activity can include at least one of thirty seconds, one minute, two minutes, three minutes, five minutes, ten minutes, and other similar periods of time.

As further shown in FIG. 6, if the processor does not determine 608 that a patient has met the initiating criteria, the processor can continue to receive 602 the patient specific information and monitor the patient as the patient goes about their daily activities as described above. Conversely, if the processor does determine 608 that the patient has satisfied the initiating criteria, the processor can invite 610 the patient to participate in the physical exertion test. For example, the processor can cause a user interface of the wearable medical device to display a notification to the patient of the initiating criteria being met. The notification can further include a query to the patient as to whether they wish to participate in the physical exertion test. If the processor determines 612 that the patient has not accepted the invitation to participate in the physical exertion test the processor can wait 614 a predetermined period of time. For example, the processor can wait 5 minutes, 10 minutes, 15 minutes, 30 minutes, one hour, two hours, 4 hours, 12 hours, one day, and other similar periods of time before continuing process 600 to initiate a physical exertion test. In certain examples, the predetermined period of time can be set by either the patient and/or the patient's health care provider when setting up the wearable medical device. In other examples, that predetermined period of time can be a default period of time programmed when the wearable medical device is initially manufactured or otherwise programmed by its manufacturer.

Referring back to FIG. 6, if the processor determines 612 that the patient has accepted the invitation to participate in the physical exertion test, the processor can initiate 616 the physical exertion test as described herein. For example, initiating 616 the physical exertion test can include prompting the patient to perform a particular activity for a predetermined period of time.

In certain implementations, the physical exertion tests as described herein can be administered in response to a prescription for a patient as provided by a healthcare professional providing care to the patient. For example, a physician may prescribe at least one physical exertion test be performed by the patient each day. In such an example, specific information such as exercise type, expected duration, initiating criteria information, sustaining criteria information, terminating criteria information, and other information related to the physical exertion test can be provided by the physician using, in certain implementations, a physician's portal or other similar computer interface.

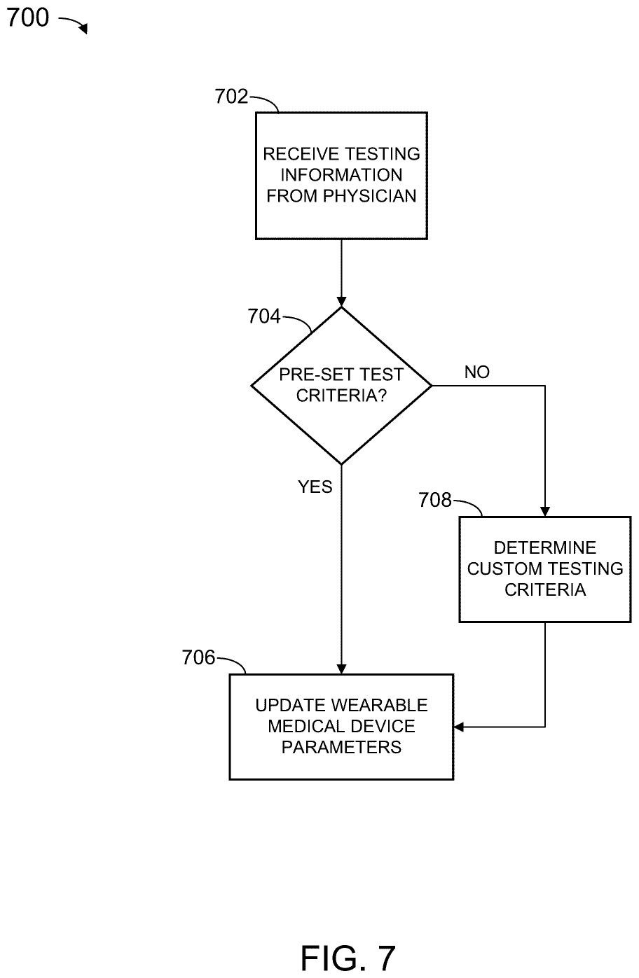

For example, as shown in FIG. 7, a process 700 includes a sample description of receiving prescribed physical exertion test information from a healthcare provider such as a patient's physician. The process 700 as shown in FIG. 7 and described herein can be implemented, for example, by a processing device at a remote computing device such as remote server 508 as shown in FIG. 5 and described above. The processor can be configured to execute one or more processes such as the physical exertion testing monitoring process 510 and the test generation process 512 as shown in FIG. 5 and described above.

As shown in FIG. 7, the process 700 can include the processor receiving 702 testing information from a healthcare professional such as a physician providing care to a cardiac patient. In certain implementations, the physician can select from a predetermined set of testing criteria or provide their own customized testing criteria for an individual cardiac patient. The processor can determine 704 whether the physician has selected predetermined testing criteria or has provided custom testing criteria. If the physician has selected from a predetermined set of testing criteria, the processor can update 706 the wearable medical device parameters related to the physical exertion test to include the predetermined testing criteria. For example, the processor can determine a set of default heartrate-dependent parameter criteria and activity-level parameter criteria as described herein. The processor can use the default criteria information to update 706 the wearable medical device parameters and stored criteria for physical exertion testing as described herein.

Conversely if the processor determines 704 that the physician has provided custom testing criteria, the processor can further process the information as received from the physician. For example, as shown in FIG. 7, the processor can determine 708 the custom testing criteria as provided by the physician and, based upon the custom testing criteria, update 706 the wearable medical device parameters for the physical exertion test as described herein.

As noted above in the discussion of FIG. 7, a physician can provide custom testing criteria for programming a prescribed wearable medical device. The custom testing criteria can be used to modify operation of the wearable medical device such that administering physical exertion tests as described herein can be customized to a particular patient.

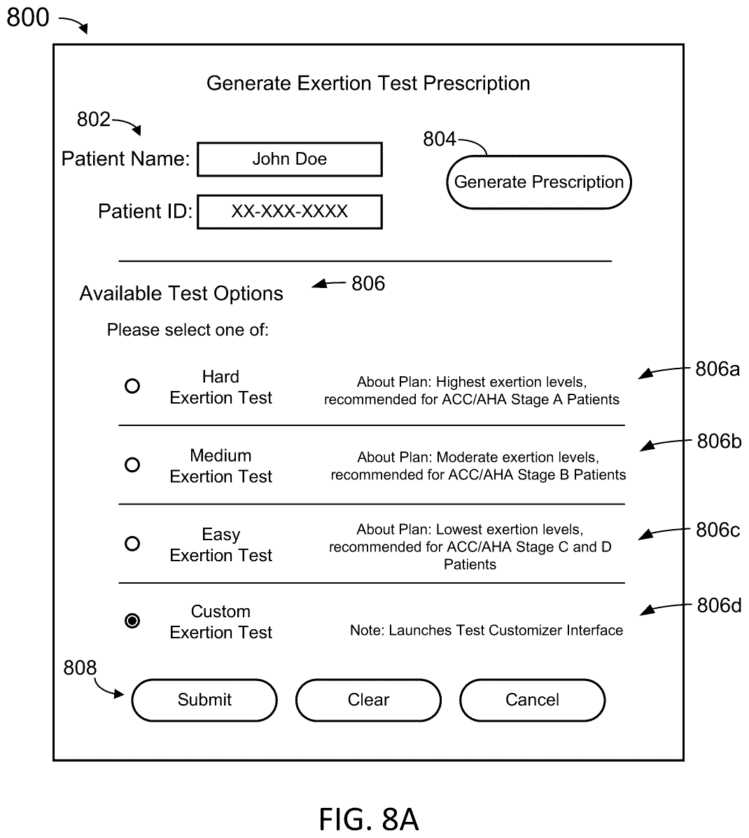

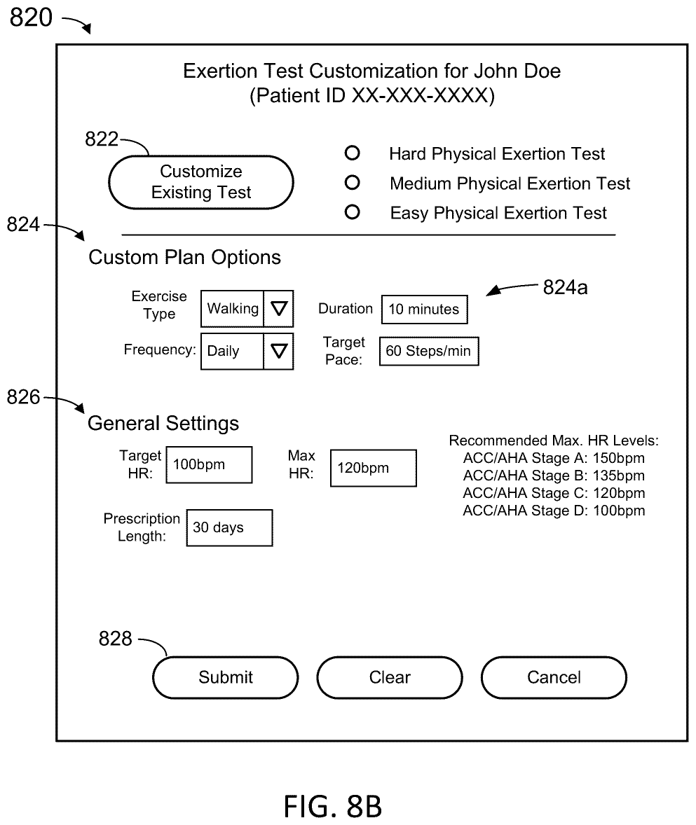

In certain implementations, the physician can access an application or other similar portal to provide information related to the physical exertion tests being prescribed to a particular patient. As shown, FIG. 8A illustrates a sample view of a user interface screen 800 that can be accessed and utilized by a physician and/or other HCP to generate an exertion test prescription for a cardiac patient. For example, the user interface screen 800 can be implemented as a portion of the plan generation process 514 as described above. The physician can access a user interface on, for example, the physician's computer 502 as described above to interact with the user interface screen 800.

As illustrated in FIG. 8A, the user interface screen 800 includes user interface controls 802, 804, 806, and 808. In some examples, the user interface control 802 provides access to patient specific information such as the cardiac patient's name and an identifier associated with the cardiac patient. In certain implementations, the patient identifier can be a medical records number associated with the cardiac patient, an insurance identification number associated with the cardiac patient, a number that directly identifies the cardiac patient such as a social security number, or another similar identification number. Based upon the patient specific information, the processor can access the cardiac patient's medical record. Based upon information contained in the medical record such as patient condition information, historical patient information, demographic information, and rehabilitation baselining information, the processor can generate a set of recommended physical test exertion criteria without additional information from the physician. The user interface control 804 can include a button or other selectable control. In some examples, the processor responds to input selecting the user interface control 804 by automatically generating a prescription including physical exertion testing for the patient. This recommended physical exertion testing can be based on a testing template (e.g., a testing template selected from the one or more testing templates 516 of FIG. 5). For instance, the processor can be configured to recommend a physical exertion test from the one or more testing templates that correspond with the ACC/AHA classification of the patient.

For example, for patients falling into Stage A, the processor can recommend a “hard” physical exertion test (e.g., a test having a long duration and more strenuously activities such as running and climbing stairs as compared to the other levels of tests). For patients falling into Stage B, the processor can recommend a “medium” physical exertion test (e.g., a test having a medium duration and moderately strenuous activities such as jogging and climbing stairs as compared to the other levels of tests). For patients falling into Stage C or Stage D, the processor can recommend an “easy” physical exertion test (e.g., a test having a short duration and less strenuous activities such as walking as compared to the other levels of tests). FIG. 8A illustrates a comparison of “easy,” “medium,” and “hard” physical exertion tests.

Additionally or alternatively, the user interface control 806 can provide access to a set of available test controls 806a-806d. As shown, the set of available test controls can include, for example, a list of available physical exertion test options from which the physician can select. For example, as shown in FIG. 8A, the set of available exertion test controls 806a-806d includes a hard test control 806a, a medium test control 806b, an easy test control 806c, and a custom test control 806d. Each of the set of available test controls 806a-806d can further include one or more notes. For example, hard test control 806a can include notes indicating that the “hard” test requires the highest initial exertion levels and is recommended for ACC/AHA Stage A patients, medium test control 806b can include notes indicating that the “medium” plan has moderate initial exertion levels and is recommended for ACC/AHA Stage B patients, easy test control 806c can include notes indicating the “easy” plan has the lowest initial exertion levels (as compared to, for example, the “medium” and “hard” plans) and is recommended for ACC/AHA Stage C and Stage D patients, the custom test control 806d can include a note indicating that selection of that control launches a plan customizer interface, which is described below in the discussion of FIG. 8B.

As shown in FIG. 8A, the user interface control 808 includes a set of selectable buttons. In response to receiving a selection of the “submit” button, the processor can generate a set of physical exertion testing criteria for the patient to be prescribed by the physician as described herein. In response to receiving a selection of the “clear” button, the processor can delete existing selections and/or entered information from the user interface screen 800. In response to receiving a selection of the “cancel” button, the processor can abort testing criteria generation.

FIG. 8B illustrates a sample user interface screen 820 that the processor can provide to a physician in response to, for example, receiving a selection of the plan control 806d of FIG. 8A by the physician. For example, the user interface screen 820 can be implemented as a portion of the testing generation process 512 as described above. The processor can provide the user interface screen 800 to interact with the physician via the physician's computer 502 of FIG. 5 as described above.

As illustrated in FIG. 8B, the user interface screen 820 includes user interface controls 822, 824, 826, and 828. In some examples, the user interface control 822 provides a physician with the option to customize an existing set of testing criteria. In certain implementations, the user interface control 822 can provide the physician with the option to select from one of a “hard” physical exertion test, a “medium” physical exertion test, or an “easy” physical exertion test to customize. Upon selection of a test and the user interface control 822, the processor can present the physician with one or more options to alter at least a portion of a physical exertion test.

Alternatively, the user interface control 824 can provide the physician with the option to create a completely custom and individualized physical exertion test for a cardiac patient. For example, as shown in FIG. 8B, the user interface control 824 can display one or more specific parameters for a physical exertion test to the physician for altering and/or updating. For example, the user interface control 824 can provide a set of parameters 824a to the physician. The parameters 824a can represent specific details related to a physical exertion test. In this example, the physician selects walking from a drop-down list of available exercise types provided within the user interface control 824 for the activity type of the physical exertion test. In some examples, this selection allows for the physician to limit what types of tests are administered to a particular patient. For example, if the patient is an ACC/AHA Stage D patient, the physician may want to limit the activity type to only walking. Conversely, if the patient is an ACC/AHA Stage A patient, the physician may want to select a set of activity types such as walking, running, jogging, and climbing stairs. The processor can receive this selection and execute subsequent processing based on the selection. For instance, in certain implementations, depending upon the type of exercises selected, the processor can display one or more additional fillable fields on user interface screen 800 for completion by the physician. As shown in FIG. 8B, the set of parameters 824a can include frequency, duration, and target pace. However, it should be noted that these individual parameters are shown by way of example only. In some examples, the set of parameters 824a can include fewer or additional parameters as needed to provide adequate information for generating a custom physical exertion test. For example, as shown in FIG. 8B, the physician selects, and the processor receives, a frequency of daily, a duration of 10 minutes, and a target pace of 60 steps per minute for parameters 824a.

It should be noted that walking is provided by way of example only. In certain implementations, the activity type for a physical exertion test can include a walking test, a jogging test, a running test, and/or a stair climbing test.

In addition to the custom and individualized plan options related to user interface control 824, the user interface screen 820 can also provide the physician with the option to provide one or more general settings options using interface controls 826. For example, the general settings options can apply to the individual criteria associated with a physical exertion test. For example, as shown in FIG. 8B, the user interface controls 826 can provide the physician with an option to provide heart rate information such as target heart rate and maximum heart rate. As shown in FIG. 8B, the physician has provided 100 bpm for a target heart rate and 120 as a maximum heart rate. For added convenience, the user interface screen 820 can further provide a note regarding recommended maximum heart rate levels. For example, as shown, the note can provide a reminder that ACC/AHA Stage A patients can have a recommended maximum heart rate of 150 bpm, ACC/AHA Stage B patients can have a recommended maximum heart rate of 135 bpm, ACC/AHA Stage C patients can have a recommended maximum heart rate of 120 bpm, ACC/AHA Stage D patients can have a recommended maximum heart rate of 100 bpm.

Additionally, the user interface control 826 can provide additional general setting controls such as a prescription length control. This control can be configured to provide a total length of time that the cardiac patient will be expected to perform the physical exertion tests. For example, as shown in FIG. 8B, the physician selects, and the processor receives, a prescription period length of 30 days. In certain implementations, the physician can select any length for the prescription period. In some implementations, the physician can select from a set of prescription periods including, for example, periods between about 2 days and about at least one of 1 week, 2 weeks, 4 weeks, 3 months, 6 months, one year, two years, and three years.

As shown in FIG. 8B, the user interface control 828 includes a set of selectable buttons. Upon completion of the custom test options and the general settings options, the physician can select one of the buttons included in the user interface control 828. In response to receiving a selection of the “submit” button, the processor can generate a customized prescription including personalized physical exertion test information such as initiating, sustaining, and terminating criteria for the patient as described herein. In response to receiving a selection of the “clear” button, the processor can delete existing selections and/or entered information from the user interface screen 820. In response to receiving a selection of the “cancel” button, the processor can abort the customized physical exertion test generation.

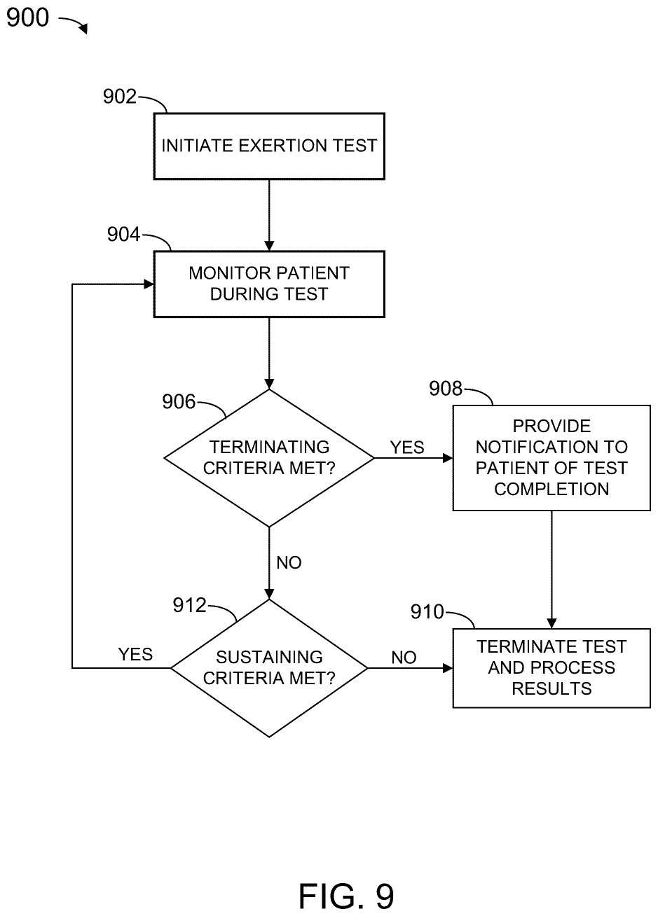

As noted above, the processor of a wearable medical device, such as processor 318 as shown in FIG. 3 and described herein, can be configured to monitor a patient and, upon the patient satisfying one or more initiating criteria, initiate a exertion test for the patient and monitor the patient's progress through the exertion test. Additionally, the processor can be further configured to monitor the patient as they perform the physical exertion test. For example, FIG. 9 illustrates process 900 for determining whether to continue or to terminate a physical exertion test based upon monitoring of the patient performing the test. For example, as shown in process 900, the processor can initiate 902 the exertion test. The processor can monitor 904 the patient during the test including, for example, monitoring heartrate-dependent parameters and activity level-dependent parameters for the patient during the physical exertion test.

Throughout the physical exertion test, the processor can determine 906 whether a terminating criteria has been met for ending the physical exertion test. Depending upon the test, the terminating criteria can vary. For example, the terminating criteria can include one or more of a total distance walked, a total distance run, a total number of stairs climbed, and/or a total time elapsed. If the processor does determine 906 that the terminating criteria has been met, the processor can provide 908 a notification to the patient of test completion. Following the notification, the processor can further terminate 910 the test and process the results of the physical exertion test for the patient. For example, processing the results can include formatting the results into a data structure and transmitting the data structure to, for example, a remote processing device for further review and analysis as described herein.

If the processor does not determine 906 that the terminating criteria has been met, the processor can further determine 912 whether sustaining criteria for continuing the physical exertion test has been met. For example, the processor can determine 912 if the physical condition of the patient allows the patient to continue the physical exertion test. More specifically, the processor can determine 912 if the patient's current heart rate falls within an allowable range for continuing the physical exertion test. For example, the sustaining criteria can include at least ones of the following acceptable heartrate ranges for continuing the physical exertion test: 60 bpm-100 bpm, 60 bpm-120 bpm, 60 bpm-135 bpm, 60 bpm-150 bpm. In some example, the sustaining criteria can include an upper heartrate limit rather than an acceptable heartrate range. In such an example, if the processor determines that the patient's heartrate is blow a certain threshold, the patient can continue the physical exertion test. For example, the heartrate threshold can include 100 bpm, 120 bpm, 125 bpm, 130 bpm, 135 bpm, 140 bpm, 145 bpm, and 150 bpm.

In some other examples, the sustaining criteria can include a minimum level of activity being maintained by the patient during the physical exertion test. For example, the sustaining criteria can include a minimum number of steps per minute, a minimum distance traveled per minute, a minimum number of stairs climbed per minute, and other similar levels of activity.

If the processor determines 912 that the sustaining criteria has been met, the processor can continue to monitor 904 the patient during the physical exertion test period conversely, if the processor determines 912 that the sustaining criteria has not been met, the processor can terminate 910 the test and process the patient's results as described herein.

In some examples as described herein, the processor can be further configured to provide information to the patient during the physical exertion test. For example, the processor can be configured to provide messages and/or notifications to the patient while monitoring 904 the patient during the physical exertion test. In some examples, the messages can include one or more of a indication to increase their pace, an indication to decrease their pace, an indication to maintain their current pace, an indication of time remaining in the physical exertion test, and/or an indication of elapsed time. Additionally, the notification can include performance information related to the patient's performance during the physical activity test. For example, the performance information can include one or more of total steps taken during the test, total distance covered during the test, total stairs climbed/descended during the test, average heartrate during the test, maximum heartrate during the test, minimum heartrate during the test, and other similar patient performance information.

Additionally, the processor can be configured to monitor 904 the patient for additional information beyond patient performance information related to the physical exertion test. For example, the processor can be configured to monitor the patient's physiological signals to determine if the patient is experiencing a cardiac arrhythmia. If the processor does determine that the patient is experiencing a cardiac arrhythmia, the processor can terminate the physical exertion test and begin treatment of the patient. Similarly, the processor can monitor the patient motion information to determine if the patient experiences a fall event during the physical exertion test. If the patient does experience a fall, the processor can be configured to terminate the physical exertion test.

In certain implementations, the processor can be configured to perform various additional tasks based upon patient monitoring. For example, if the processor determines that the patient is experiencing a cardiac arrhythmia, the processor can provide one or more outputted alerts prior to treatment of the patient. For example, the alerts can include an audio and/or visual alert to the patient and any bystanders that may be close by, the alert indicating that the patient is experiencing a cardiac arrhythmia. The alert can further include a notification to stand back a certain distance (e.g., six feet) from the patient during treatment. Similarly, the one or more outputted alerts can include an electronic notification (e.g., a text message, an email, a digital alert issued through one or more caregiver portal applications) to a doctor or other similar caregiver associated with the patient that the patient has experienced a cardiac arrhythmia and is receiving (or has received) treatment.

When monitoring a patient experiencing a fall event, the processor can monitor the output of, for example, one or more three-axis accelerometers as described herein (e.g., accelerometers 322 as described above). By monitoring the output of the accelerometers for a specific force vector pattern, the processor can determine whether the patient has experienced a fall event.

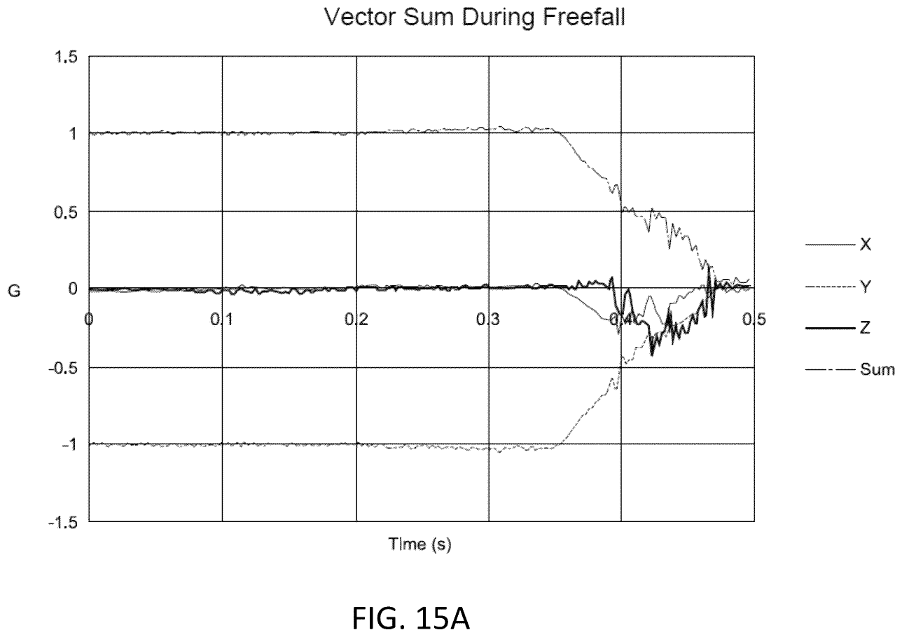

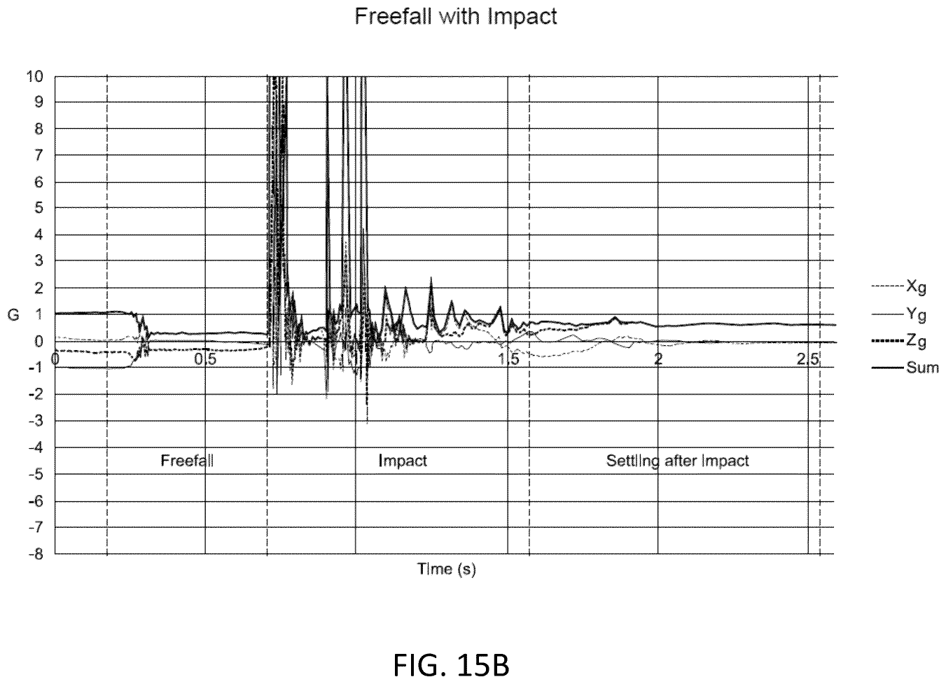

For example, FIG. 15A is a graph of forces that acted on a patient as a function of time during an unsupported fall event where the patient falls to the ground (or to another surface that interrupts the patient's movement during the fall event). The forces shown are oriented in x, y, and z directions in a Cartesian coordinate system. A sum of the absolute value of these forces is also shown. While the example data shown in FIG. 15A was collected from a three-axis accelerometer, it is appreciated that alternative representations of the physical phenomena of an unsupported fall of a patient to the ground are possible depending on the sensor(s) used and the orientation of the sensors. The positive and negative sign of the data depicted in FIG. 15A was a function of the orientation of the three-axis accelerometer. For the purposes of explanation, only the magnitude as a function of time will be discussed.

As shown, at Time (t)=0 seconds(s), a patient was standing upright. Resisting the force of gravity, the patient (or more specifically the accelerometer attached to the patient) experienced a vertical force equal to 1G (in the negative y direction according to the orientation of the accelerometer on the patient). This force of 1G continued until approximately t=0.35 s, at which point the patient began an unsupported fall to the ground (also known as “free fall”). During freefall from t=0.35 s to approximately t=0.48 s, the net force acting on the patient declined because the patient was no longer resisting the pull of gravity by standing upright. This decline continued until the patient made contact with the ground. The various forces acting on the patient become approximately zero just before the patient impacted the ground.

FIG. 15B displays data similar to the data shown in FIG. 15A but over a longer timescale. FIG. 15B further illustrates the forces experienced by the patient during and after impact with the ground. As in FIG. 15B, a patient started out in a stable state with a net force acting on the patient of approximately 1G. As the patient freefell toward the ground, the forces declined for the same reasons explained above. However, as indicated in the region labeled “Impact,” once the patient made contact with the ground, the forces of impact were over 10G. In the example shown, there were successive waves of high force as the patient bounces on the ground and as the forces of impact reverberated throughout the body of the patient. The impact forces eventually dissipated, as is shown in the region labeled “Settling after impact.”

By monitoring the output of one or more accelerometers for force information such as that shown in FIGS. 15A and 15B, the processor can monitor for a patient fall event. If the processor does determine that the patient has experienced a fall event, the processor can further determine if the patient is experiencing a cardiac arrhythmia. If the patient is experiencing a cardiac arrhythmia, the processor can output one or more alerts as noted above and begin treatment of the patient. If the patient has not experienced a cardiac arrythmia, the processor can output one or more alerts about the fall event (e.g., to a doctor or caregiver associated with the patient).

As described herein, a wearable medical device can include a user interface such as user interface 308 as shown in FIG. 3 and described above. In certain implementations, a processor such as processor 318 can be configured to provide notifications to a patient using the user interface as well as to receive information from the patient. For example, as the patient goes about their normal activities in their daily routines, the processor can provide various notifications to the patient. In certain implementations, if the processor determines that the patient is performing a particular physical activity, or the processor is otherwise scheduled to administer a physical exertion test, the processor can provide a notification to the patient via the user interface and prompt the user to participate in a physical exertion test. More specifically, the processor can provide information related to the physical exertion tests as described herein and receive feedback information from the patient prior to, during, and after the physical exertion tests.

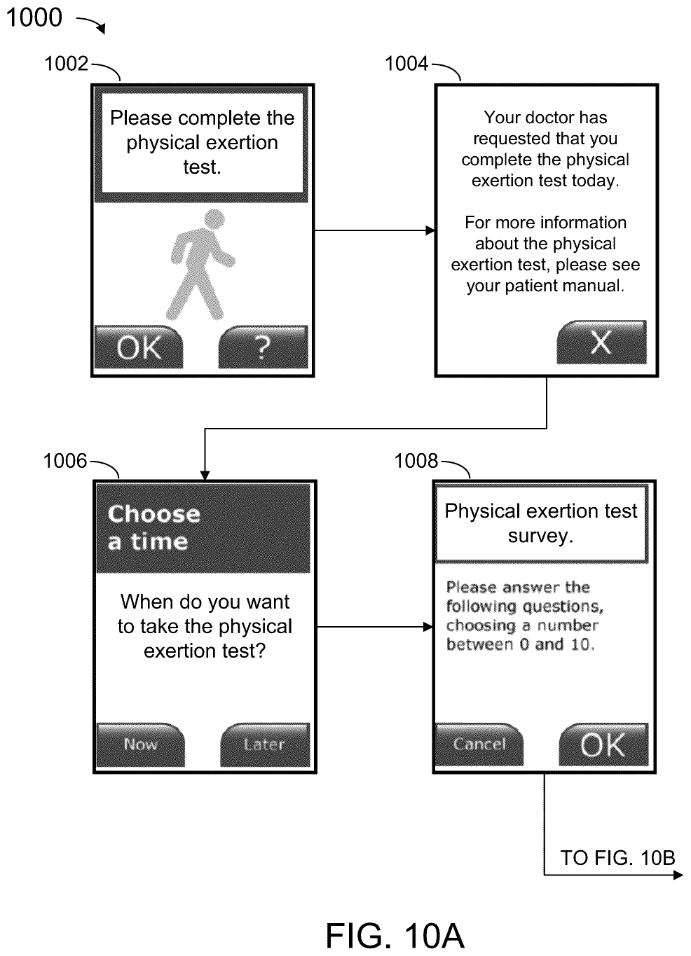

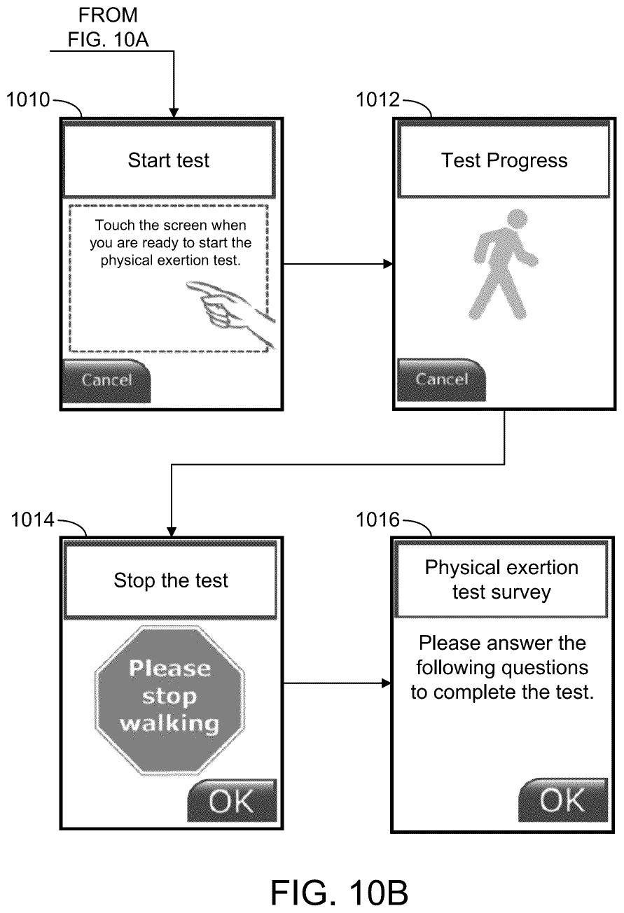

For example, FIGS. 10A and 10B combine to illustrate a sample screenshot process flow 1000. The process flow 1000 includes a set of sample screenshots that a processor can display on a user interface to provide information to and receive information from a patient prior to, during, and after a physical exertion test as described herein. As shown in FIG. 10A, the processor can provide a notification to the patient that they are to complete a physical exertion test as shown, for example, in screenshot 1002. As further shown in FIG. 10A, screenshot 1004 includes additional information to the patient regarding the purpose of the physical exertion test period for example, the additional information indicates that the patient's physician has requested that they complete the physical exertion test. The user interface can update to display subsequent screenshot 1006. for example, screenshot 1006 includes a query to the patient regarding when the patient would like to complete the physical exertion test. In certain implementations, as described herein, the patient has the option to delay the physical exertion test for a period of time or to immediately begin the physical exertion test. If the patient indicates they would like to begin the physical exertion test, the processor can further provide a survey of questions to the patient prior to initiating the physical exertion test. For example, as shown in screenshot 1008, the patient can be prompted to complete a survey prior to beginning the physical exertion test. For example, the survey can include questions regarding the patient's current physical states such as questions related to the patient's pain levels, fatigue levels, shortness of breath levels, and questions about other similar physical conditions.

Moving to FIG. 10 B, the processor can display and indication that the device is ready to begin monitoring the patient during the physical exertion test period for example, screenshot 1010 includes an indication that the device is ready to start the physical exertion test and a request for the patient to interact with the user interface to begin the test. As the patient performs the physical exertion test, the processor can continually update the user interface to provide an indication of the patient status during the physical exertion test period for example, as shown in screenshot 1012, the user interface can be updated to provide an indication of the patient's progression through the physical exertion test period upon completion of the physical exertion test, the processor can provide an indication to the user via the user interface to stop the physical activity. For example, screenshot 1014 provides an indication to the user to stop performing the activity as the physical exertion test has completed. The processor can further provide a survey of questions to the patient following conclusion of the physical exertion test period for example, as shown in screenshot 1016, the patient can be prompted to completes a survey following completion of the physical exertion test period. For instance, the survey can include questions regarding the patience current physical state following the physical exertion test such as, for example, questions related to the patient's pain levels, fatigue levels, shortness of breath levels, and questions about other similar physical conditions for the patient.

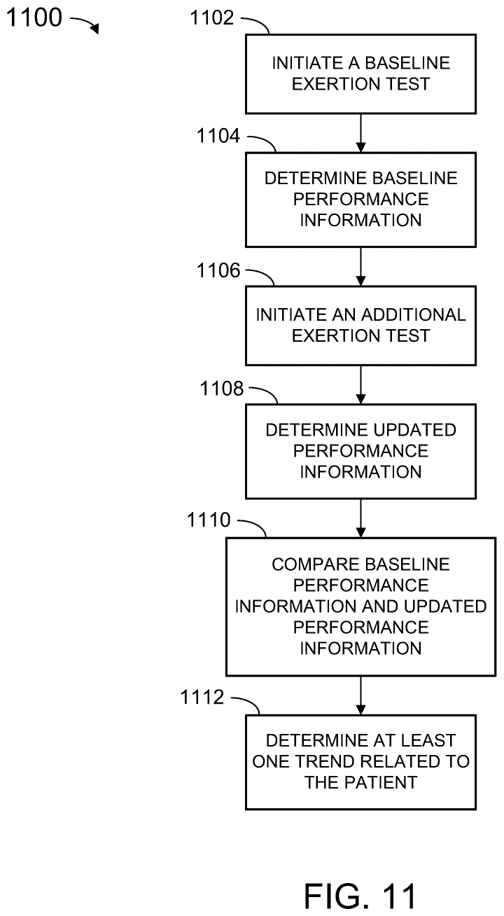

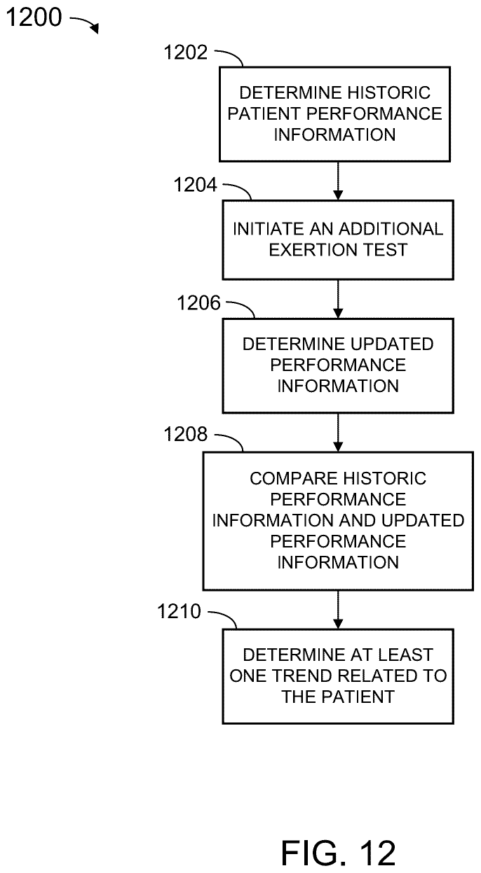

Based upon information collected during a physical exertion test, a processing device can determine information related to the health, or information related to changes in the health, of the patient performing the physical exertion test. For example, a processor of the remote server 508 as shown in FIG. 5 and described above, can be configured to receive and process information related to a patient's performance during a physical exertion test. FIGS. 11 and 12 illustrate sample processes for processing received information and determining additional information related to a patient performing a physical exertion test.

For example, as shown in FIG. 11, process 1100 can include comparing updated patient performance information to baseline patient performance information to determine changes in the cardiac condition or health of a cardiac patient in, for example, a different environment such as the patient's home. For example, as shown in FIG. 11, a processor can initiate 1102 a baseline exertion test for a patient. For example, the baseline exertion tests can include a test performed in a monitored environment such as during a hospital stay or in a healthcare provider's office by a cardiac patient.

The processor can process the information as obtained during the baseline exertion test to determine 1104 baseline performance information for the cardiac patient. Over time, the processor can initiate 1106 an additional physical exertion test to be performed by the cardiac patient. Following the additional physical exertion test, the processor can determine 1108 updated performance information for the patient as collected during the additional physical exertion test.

The processor can further compare 1110 the baseline performance information against the updated performance information to determine any changes in the information. Based upon the changes in the information, the processor can determine 1112 at least one trend related to the cardiac patient, the trend indicating a change in the overall condition of the cardiac patient. For example, if the patient's average heartrate as measured during the physical exertion test decreases between the baseline exertion test and the additional exertion test, the processor can determine a positive trend indicating potential cardiac improvement for the patient. The processor can be configured to format the trend and change information into a report for review by, for example, the patient's HCP.

FIG. 12 illustrates a similar process 1200 that includes comparing updated patient performance information to collected historic patient performance information to determine changes in the cardiac condition or health of a cardiac patient over time. For example, as shown in FIG. 12, a processor can determine 1202 historic performance information for the cardiac patient. Over time, the processor can collect physical exertion test information for the patient to establish a set of historic patient information including, for example, physiological information for the patient as measured during physical exertion tests (e.g., average heartrate, high heartrate, low heartrate) as well as activity information for the patient (e.g., total distance covered, steps per minute, total duration).

As further shown in FIG. 12, the processor can initiate 1204 an additional physical exertion test to be performed by the cardiac patient. Following the additional physical exertion test, the processor can determine 1206 updated performance information for the patient as collected during the additional physical exertion test.

The processor can further compare 1208 the historic performance information against the updated performance information to determine any changes in the information. Based upon the changes in the information, the processor can determine 1210 at least one trend related to the cardiac patient, the trend indicating a change in the overall condition of the cardiac patient.

In order to process the patient performance information and provide feedback (e.g., a patient report) to the patient's physician, the processor implementing the testing process can be configured to follow a set of rules. For example, when various sets of rules are satisfied, the resulting information that can be processed and provided by the processor varies accordingly. In certain implementations, the rules can analyze various aspects of the physical exertion testing procedure including, for example, patient answers to the pre-test questions, patient performance during the test, and patient answers to the post-test questions. For example, if the patient provides unsatisfactory answers to the pre-test questions, the processor can cancel the test and report accordingly. Similarly, if the patient stops performing the physical activity in the middle of the exertion test, the processor can provide an indication that the test was started but not completed. The following listing provides various rule scenarios and potential results that can be compiled by the processor in the post physical exertion test report.
Scenario: Patient provides satisfactory answers for pre-test questions. The patient completes the test. The patient provides satisfactory answers to the post-test questions.
Result: The report includes data and graph information displaying heartrate and step information during the test. Answers to pre-test and post-test questions also included. No warnings displayed.
Scenario: Patient provides satisfactory answers for pre-test questions. The patient completes the test. The patient provides unsatisfactory or no answers to the post-test questions.
Result: The report includes data and graph information displaying heartrate and step information during the test. Answers to pre-test questions are included. No answers to the post-test questions included if left blank and no warnings displayed. Unsatisfactory answers to the post-test questions included if provided and one or more warnings can be displayed.
Scenario: Patient provides satisfactory answers for pre-test questions. The patient does not complete the test. The patient is not presented post walk questions
Result: The report includes data and graph information displaying heartrate and step information during the completed portion of the test. A warning that the walk test was incomplete will be included. There will be no answers for the post-test questions.
Scenario: Patient provides unsatisfactory answers for one or more pre-test questions. The patient does not get prompted to do the walk test. The patient is not presented post walk questions
Result: No information related to patient test performance is displayed. A warning that the patient answered unsatisfactorily to the pre-test questions is included. There will be no answers for the post-test questions.

In certain implementations, the information contained in the report (e.g., graph data showing heartrate and distance information) can also be updated to include determined trend information. This information can be provided to the physician as a separate trends analysis that is formatted according to a similar set of rules as above. For example, if the patient completes the physical exertion test and provides answers to the post-test questions, the trends data can include an updated analysis of patient trends information based upon both heartrate and step information as completed during the test. If the patient completes the physical exertion test but does not answer the post-test questions, the trends data can still include an analysis of the heartrate and step information as completed during the test. If the patient completes the physical exertion test but there is no heartrate information recorded, the trends data can be updated based upon the step information and heartrate information can be estimated or otherwise included but marked as incomplete (e.g., represented by a broken line in the graph). If the patient starts the physical exertion test but does not complete the test, complete heartrate and step information can be estimated based upon the average heartrate and step information as collected during the portion of the physical exertion test that was completed. If the patient did not perform any portion of the physical exertion test, the graph can include a break on the graph to provide an indication of the testing date and that no test was completed to maintain uniform data reporting/representing in the trends data. Similarly, the patient's answers to the pre-test questions and the post-test questions can be included in one or more reports associated with the trends data analysis.

In some examples, to provide for consistent and uniform reporting and trends analysis, a single physical exertion test can be administered per day for a particular patient. However, in some examples, multiple physical exertion tests can be administered in a single day for the same patient. For example, provided that the patient provides satisfactory answers to pre-test and post-test questions, and no abnormal cardiac events are detected in the patient's physiological signals during the continuous cardiac monitoring, the patient can be prompted to participate in multiple physical exertion tests in the same day.

The teachings of the present disclosure can be generally applied to external medical monitoring and/or treatment devices that include one or more accelerometers as described herein. Such external medical devices can include, for example, ambulatory medical devices as described herein that are capable of and designed for moving with the patient as the patient goes about his or her daily routine. An example ambulatory medical device can be a wearable medical device such as a WCD, a wearable cardiac monitoring device, an in-hospital device such as an in-hospital wearable defibrillator (HWD), a short-term wearable cardiac monitoring and/or therapeutic device, mobile cardiac event monitoring devices, and other similar wearable medical devices.

The wearable medical device can be capable of continuous use by the patient. In some implementations, the continuous use can be substantially or nearly continuous in nature. That is, the wearable medical device can be continuously used, except for sporadic periods during which the use temporarily ceases (e.g., while the patient bathes, while the patient is refit with a new and/or a different garment, while the battery is charged/changed, while the garment is laundered, etc.). Such substantially or nearly continuous use as described herein may nonetheless be considered continuous use. For example, the wearable medical device can be configured to be worn by a patient for as many as 24 hours a day. In some implementations, the patient can remove the wearable medical device for a short portion of the day (e.g., for half an hour to bathe).

Further, the wearable medical device can be configured as a long term or extended use medical device. Such devices can be configured to be used by the patient for an extended period of several days, weeks, months, or even years. In some examples, the wearable medical device can be used by a patient for an extended period of at least one week. In some examples, the wearable medical device can be used by a patient for an extended period of at least 30 days. In some examples, the wearable medical device can be used by a patient for an extended period of at least one month. In some examples, the wearable medical device can be used by a patient for an extended period of at least two months. In some examples, the wearable medical device can be used by a patient for an extended period of at least three months. In some examples, the wearable medical device can be used by a patient for an extended period of at least six months. In some examples, the wearable medical device can be used by a patient for an extended period of at least one year. In some implementations, the extended use can be uninterrupted until a physician or other HCP provides specific instruction to the patient to stop use of the wearable medical device.

Regardless of the extended period of wear, the use of the wearable medical device can include continuous or nearly continuous wear by the patient as described above. For example, the continuous use can include continuous wear or attachment of the wearable medical device to the patient, e.g., through one or more of the electrodes as described herein, during both periods of monitoring and periods when the device may not be monitoring the patient but is otherwise still worn by or otherwise attached to the patient. The wearable medical device can be configured to continuously monitor the patient for cardiac-related information (e.g., ECG information, including arrhythmia information, cardio-vibrations, etc.) and/or non-cardiac information (e.g., blood oxygen, the patient's temperature, glucose levels, tissue fluid levels, and/or lung vibrations). The wearable medical device can carry out its monitoring in periodic or aperiodic time intervals or times. For example, the monitoring during intervals or times can be triggered by a user action or another event.

As noted above, the wearable medical device can be configured to monitor other physiologic parameters of the patient in addition to cardiac related parameters. For example, the wearable medical device can be configured to monitor, for example, pulmonary-vibrations (e.g., using microphones and/or accelerometers), breath vibrations, sleep related parameters (e.g., snoring, sleep apnea), tissue fluids (e.g., using radio-frequency transmitters and sensors), among others.

Other example wearable medical devices include automated cardiac monitors and/or defibrillators for use in certain specialized conditions and/or environments such as in combat zones or within emergency vehicles. Such devices can be configured so that they can be used immediately (or substantially immediately) in a life-saving emergency. In some examples, the ambulatory medical devices described herein can be pacing-enabled, e.g., capable of providing therapeutic pacing pulses to the patient. In some examples, the ambulatory medical devices can be configured to monitor for and/or measure ECG metrics including, for example, heartrate (such as average, median, mode, or other statistical measure of the heartrate, and/or maximum, minimum, resting, pre-exercise, and post-exercise heartrate values and/or ranges), heartrate variability metrics, PVC burden or counts, atrial fibrillation burden metrics, pauses, heartrate turbulence, QRS height, QRS width, changes in a size or shape of morphology of the ECG information, cosine R-T, artificial pacing, QT interval, QT variability, T wave width, T wave alternans, T-wave variability, and ST segment changes.

As noted above, FIG. 3 illustrates an example component-level view of a medical device controller 300 included in, for example, a wearable medical device. As further shown in FIG. 3, the therapy delivery circuitry 302 can be coupled to one or more electrodes 320 configured to provide therapy to the patient. For example, the therapy delivery circuitry 302 can include, or be operably connected to, circuitry components that are configured to generate and provide an electrical therapeutic shock. The circuitry components can include, for example, resistors, capacitors, relays and/or switches, electrical bridges such as an h-bridge (e.g., including a plurality of insulated gate bipolar transistors or IGBTs), voltage and/or current measuring components, and other similar circuitry components arranged and connected such that the circuitry components work in concert with the therapy delivery circuitry and under control of one or more processors (e.g., processor 318) to provide, for example, at least one therapeutic shock to the patient including one or more pacing, cardioversion, or defibrillation therapeutic pulses.

Pacing pulses can be used to treat cardiac arrhythmia conditions such as bradycardia (e.g., less than 30 beats per minute) and tachycardia (e.g., more than 150 beats per minute) using, for example, fixed rate pacing, demand pacing, anti-tachycardia pacing, and the like. Defibrillation pulses can be used to treat ventricular tachycardia and/or ventricular fibrillation.

The capacitors can include a parallel-connected capacitor bank consisting of a plurality of capacitors (e.g., two, three, four or more capacitors). In some examples, the capacitors can include a single film or electrolytic capacitor as a series connected device including a bank of the same capacitors. These capacitors can be switched into a series connection during discharge for a defibrillation pulse. For example, a single capacitor of approximately 140 uF or larger, or four capacitors of approximately 650 uF can be used. The capacitors can have a 1600 VDC or higher rating for a single capacitor, or a surge rating between approximately 350 to 500 VDC for paralleled capacitors and can be charged in approximately 15 to 30 seconds from a battery pack.

For example, each defibrillation pulse can deliver between 60 to 180 joules of energy. In some implementations, the defibrillating pulse can be a biphasic truncated exponential waveform, whereby the signal can switch between a positive and a negative portion (e.g., charge directions). This type of waveform can be effective at defibrillating patients at lower energy levels when compared to other types of defibrillation pulses (e.g., such as monophasic pulses). For example, an amplitude and a width of the two phases of the energy waveform can be automatically adjusted to deliver a precise energy amount (e.g., 150 joules) regardless of the patient's body impedance. The therapy delivery circuitry 302 can be configured to perform the switching and pulse delivery operations, e.g., under control of the processor 318. As the energy is delivered to the patient, the amount of energy being delivered can be tracked. For example, the amount of energy can be kept to a predetermined constant value even as the pulse waveform is dynamically controlled based on factors such as the patient's body impedance which the pulse is being delivered.

In certain examples, the therapy delivery circuitry 302 can be configured to deliver a set of cardioversion pulses to correct, for example, an improperly beating heart. When compared to defibrillation as described above, cardioversion typically includes a less powerful shock that is delivered at a certain frequency to mimic a heart's normal rhythm.

The data storage 304 can include one or more of non-transitory computer-readable media, such as flash memory, solid state memory, magnetic memory, optical memory, cache memory, combinations thereof, and others. The data storage 304 can be configured to store executable instructions and data used for operation of the medical device controller 300. In certain examples, the data storage can include executable instructions that, when executed, are configured to cause the processor 318 to perform one or more operations. In some examples, the data storage 304 can be configured to store information such as ECG data as received from, for example, the sensing electrode interface.

In some examples, the network interface 306 can facilitate the communication of information between the medical device controller 300 and one or more other devices or entities over a communications network. For example, where the medical device controller 300 is included in an ambulatory medical device, the network interface 306 can be configured to communicate with a remote computing device such as a remote server or other similar computing device. The network interface 306 can include communications circuitry for transmitting data in accordance with a Bluetooth® wireless standard for exchanging such data over short distances to an intermediary device. For example, such an intermediary device can be configured as a base station, a “hotspot” device, a smartphone, a tablet, a portable computing device, and/or other devices in proximity of the wearable medical device including the medical device controller 300. The intermediary device(s) may in turn communicate the data to a remote server over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with a remote server over a Wi-Fi™ communications link based on the IEEE 802.11 standard.

In certain examples, the user interface 308 can include one or more physical interface devices such as input devices, output devices, and combination input/output devices and a software stack configured to drive operation of the devices. These user interface elements can render visual, audio, and/or tactile content. Thus, the user interface 308 can receive input or provide output, thereby enabling a user to interact with the medical device controller 300.

The medical device controller 300 can also include at least one rechargeable battery 310 configured to provide power to one or more components integrated in the medical device controller 300. The rechargeable battery 310 can include a rechargeable multi-cell battery pack. In one example implementation, the rechargeable battery 310 can include three or more 2200 mAh lithium ion cells that provide electrical power to the other device components within the medical device controller 300. For example, the rechargeable battery 310 can provide its power output in a range of between 20 mA to 1000 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the medical device controller 300.

The sensor interface 312 can include physiological signal circuitry that is coupled to one or more sensors configured to monitor one or more physiological parameters of the patient. As shown, the sensors can be coupled to the medical device controller 300 via a wired or wireless connection. The sensors can include one or more ECG sensing electrodes 322, and non-ECG physiological sensors 323 such as vibration sensor 324, tissue fluid monitors 326 (e.g., based on ultra-wide band radiofrequency devices), and motion sensors (e.g., accelerometers, gyroscopes, and/or magnetometers). In some implementations, the sensors can include a plurality of conventional ECG sensing electrodes in addition to digital sensing electrodes.

The sensing electrodes 322 can be configured to monitor a patient's ECG information. For example, by design, the digital sensing electrodes 322 can include skin-contacting electrode surfaces that may be deemed polarizable or non-polarizable depending on a variety of factors including the metals and/or coatings used in constructing the electrode surface. All such electrodes can be used with the principles, techniques, devices and systems described herein. For example, the electrode surfaces can be based on stainless steel, noble metals such as platinum, or Ag—AgCl.

In some examples, the electrodes 322 can be used with an electrolytic gel dispersed between the electrode surface and the patient's skin. In certain implementations, the electrodes 322 can be dry electrodes that do not need an electrolytic material. As an example, such a dry electrode can be based on tantalum metal and having a tantalum pentoxide coating as is described above. Such dry electrodes can be more comfortable for long term monitoring applications.

Referring back to FIG. 3, the vibration sensors 324 can be configured to detect cardiac or pulmonary vibration information. For example, the vibration sensors 324 can detect a patient's heart valve vibration information. For example, the vibration sensors 324 can be configured to detect cardio-vibrational signal values including any one or all of S1, S2, S3, and S4. From these cardio-vibrational signal values or heart vibration values, certain heart vibration metrics may be calculated, including any one or more of electromechanical activation time (EMAT), average EMAT, percentage of EMAT (% EMAT), systolic dysfunction index (SDI), and left ventricular systolic time (LVST). The vibration sensors 324 can also be configured to detect heart wall motion, for instance, by placement of the sensor in the region of the apical beat. The vibration sensors 324 can include a vibrational sensor configured to detect vibrations from a subject's cardiac and pulmonary system and provide an output signal responsive to the detected vibrations of a targeted organ, for example, being able to detect vibrations generated in the trachea or lungs due to the flow of air during breathing. In certain implementations, additional physiological information can be determined from pulmonary-vibrational signals such as, for example, lung vibration characteristics based on sounds produced within the lungs (e.g., stridor, crackle, etc.). The vibration sensors 324 can also include a multi-channel accelerometer, for example, a three-channel accelerometer configured to sense movement in each of three orthogonal axes such that patient movement/body position can be detected and correlated to detected cardio-vibrations information. The vibration sensors 324 can transmit information descriptive of the cardio-vibrations information to the sensor interface 312 for subsequent analysis.

The tissue fluid monitors 326 can use radio frequency (RF) based techniques to assess fluid levels and accumulation in a patient's body tissue. For example, the tissue fluid monitors 326 can be configured to measure fluid content in the lungs, typically for diagnosis and follow-up of pulmonary edema or lung congestion in heart failure patients. The tissue fluid monitors 326 can include one or more antennas configured to direct RF waves through a patient's tissue and measure output RF signals in response to the waves that have passed through the tissue. In certain implementations, the output RF signals include parameters indicative of a fluid level in the patient's tissue. The tissue fluid monitors 326 can transmit information descriptive of the tissue fluid levels to the sensor interface 312 for subsequent analysis.

In certain implementations, the cardiac event detector 316 can be configured to monitor a patient's ECG signal for an occurrence of a cardiac event such as an arrhythmia or other similar cardiac event. The cardiac event detector can be configured to operate in concert with the processor 318 to execute one or more methods that process received ECG signals from, for example, the sensing electrodes 322 and determine the likelihood that a patient is experiencing a cardiac event. The cardiac event detector 316 can be implemented using hardware or a combination of hardware and software. For instance, in some examples, cardiac event detector 316 can be implemented as a software component that is stored within the data storage 304 and executed by the processor 318. In this example, the instructions included in the cardiac event detector 316 can cause the processor 318 to perform one or more methods for analyzing a received ECG signal to determine whether an adverse cardiac event is occurring. In other examples, the cardiac event detector 316 can be an application-specific integrated circuit (ASIC) that is coupled to the processor 318 and configured to monitor ECG signals for adverse cardiac event occurrences. Thus, examples of the cardiac event detector 316 are not limited to a particular hardware or software implementation.

In some implementations, the processor 318 includes one or more processors (or one or more processor cores) that each are configured to perform a series of instructions that result in manipulated data and/or control the operation of the other components of the medical device controller 300. In some implementations, when executing a specific process (e.g., cardiac monitoring), the processor 318 can be configured to make specific logic-based determinations based on input data received and be further configured to provide one or more outputs that can be used to control or otherwise inform subsequent processing to be carried out by the processor 318 and/or other processors or circuitry with which processor 318 is communicatively coupled. Thus, the processor 318 reacts to specific input stimulus in a specific way and generates a corresponding output based on that input stimulus. In some example cases, the processor 318 can proceed through a sequence of logical transitions in which various internal register states and/or other bit cell states internal or external to the processor 318 can be set to logic high or logic low. As referred to herein, the processor 318 can be configured to execute a function where software is stored in a data store coupled to the processor 318, the software being configured to cause the processor 118 to proceed through a sequence of various logic decisions that result in the function being executed. The various components that are described herein as being executable by the processor 318 can be implemented in various forms of specialized hardware, software, or a combination thereof. For example, the processor 318 can be a digital signal processor (DSP) such as a 24-bit DSP. The processor 318 can be a multi-core processor, e.g., having two or more processing cores. The processor 318 can be an Advanced RISC Machine (ARM) processor such as a 32-bit ARM processor or a 64-bit ARM processor. The processor 318 can execute an embedded operating system, and include services provided by the operating system that can be used for file system manipulation, display & audio generation, basic networking, firewalling, data encryption and communications.

As noted above, an ambulatory medical device such as a WCD can be designed to include a digital front-end where analog signals sensed by skin-contacting electrode surfaces of a set of digital sensing electrodes are converted to digital signals for processing. Typical ambulatory medical devices with analog front-end configurations use circuitry to accommodate a signal from a high source impedance from the sensing electrode (e.g., having an internal impedance range from approximately 100 Kiloohms to one or more Megaohms). This high source impedance signal is processed and transmitted to a monitoring device such as processor 318 of the controller 300 as described above for further processing. In certain implementations, the monitoring device, or another similar processor such as a microprocessor or another dedicated processor operably coupled to the sensing electrodes, can be configured to receive a common noise signal from each of the sensing electrodes, sum the common noise signals, invert the summed common noise signals and feed the inverted signal back into the patient as a driven ground using, for example, a driven right leg circuit to cancel out common mode signals.

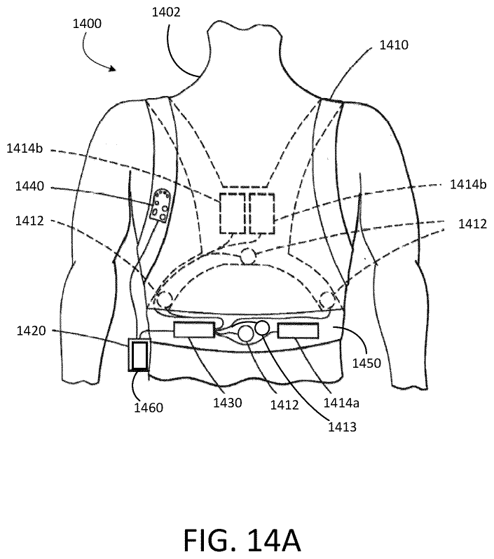

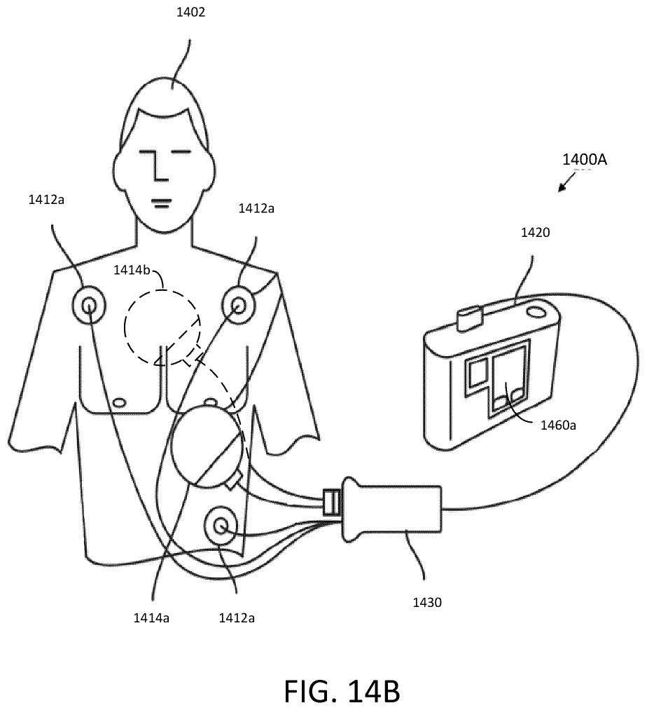

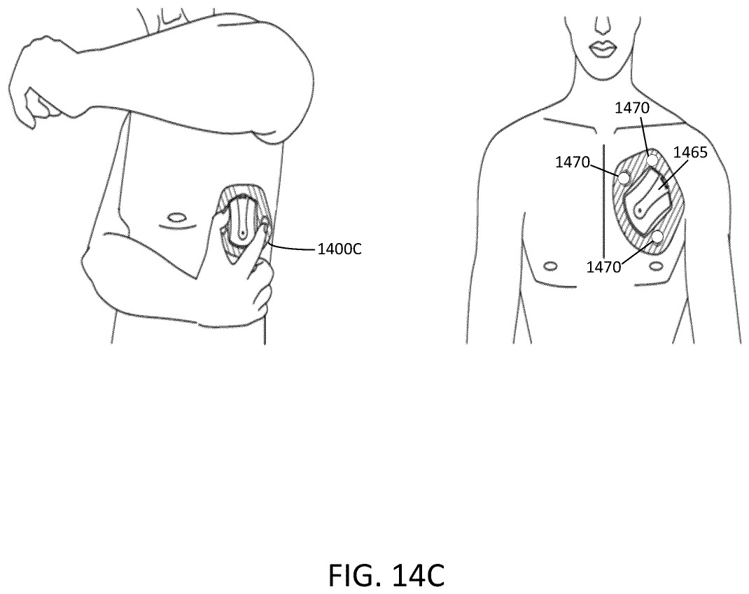

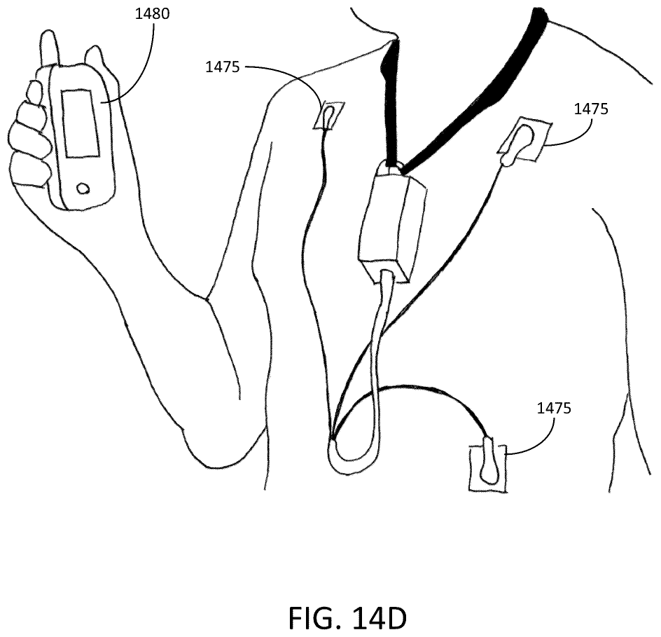

FIG. 14A illustrates an example medical device 1400 that is external, ambulatory, and wearable by a patient 1402, and configured to implement one or more configurations described herein. For example, the medical device 1400 can be a non-invasive medical device configured to be located substantially external to the patient. Such a medical device 1400 can be, for example, an ambulatory medical device that is capable of and designed for moving with the patient as the patient goes about his or her daily routine. For example, the medical device 1400 as described herein can be bodily-attached to the patient such as the LifeVest® wearable cardioverter defibrillator available from ZOLL® Medical Corporation. Such wearable defibrillators typically are worn nearly continuously or substantially continuously for two to three months at a time.

During the period of time in which they are worn by the patient, the wearable defibrillator can be configured to continuously or substantially continuously monitor the vital signs of the patient and, upon determination that treatment is required, can be configured to deliver one or more therapeutic electrical pulses to the patient. For example, such therapeutic shocks can be pacing, defibrillation, or transcutaneous electrical nerve stimulation (TENS) pulses.

The medical device 1400 can include one or more of the following: a garment 1410, one or more ECG sensing electrodes 1412, one or more non-ECG physiological sensors 1413, one or more therapy electrodes 1414a and 1414b (collectively referred to herein as therapy electrodes 1414), a medical device controller 1420 (e.g., controller 300 as described above in the discussion of FIG. 3), a connection pod 1430, a patient interface pod 1440, a belt 1450, or any combination of these. In some examples, at least some of the components of the medical device 1400 can be configured to be affixed to the garment 1410 (or in some examples, permanently integrated into the garment 1410), which can be worn about the patient's torso.

The medical device controller 1420 can be operatively coupled to the sensing electrodes 1412, which can be affixed to the garment 1410, e.g., assembled into the garment 1410 or removably attached to the garment, e.g., using hook and loop fasteners. In some implementations, the sensing electrodes 1412 can be permanently integrated into the garment 1410. The medical device controller 1420 can be operatively coupled to the therapy electrodes 1414. For example, the therapy electrodes 1414 can also be assembled into the garment 1410, or, in some implementations, the therapy electrodes 1414 can be permanently integrated into the garment 1410. In an example, the medical device controller 1420 includes a patient user interface 1460 to allow a patient interface with the externally-worn device. For example, the patient can use the patient user interface 1460 to respond to activity related questions, prompts, and surveys as described herein.

Component configurations other than those shown in FIG. 14A are possible. For example, the sensing electrodes 1412 can be configured to be attached at various positions about the body of the patient 1402. The sensing electrodes 1412 can be operatively coupled to the medical device controller 1420 through the connection pod 1430. In some implementations, the sensing electrodes 1412 can be adhesively attached to the patient 1402. In some implementations, the sensing electrodes 1412 and at least one of the therapy electrodes 1414 can be included on a single integrated patch and adhesively applied to the patient's body.

The sensing electrodes 1412 can be configured to detect one or more cardiac signals. Examples of such signals include ECG signals and/or other sensed cardiac physiological signals from the patient. In certain examples, as described herein, the non-ECG physiological sensors 1413 such as accelerometers, vibrational sensors, and other measuring devices for recording additional non-ECG physiological parameters. For example, as described above, the such non-ECG physiological sensors are configured to detect other types of patient physiological parameters and acoustic signals, such as tissue fluid levels, cardio-vibrations, lung vibrations, respiration vibrations, patient movement, etc.

In some examples, the therapy electrodes 1414 can also be configured to include sensors configured to detect ECG signals as well as other physiological signals of the patient. The connection pod 1430 can, in some examples, include a signal processor configured to amplify, filter, and digitize these cardiac signals prior to transmitting the cardiac signals to the medical device controller 1420. One or more of the therapy electrodes 1414 can be configured to deliver one or more therapeutic defibrillating shocks to the body of the patient 1402 when the medical device 1400 determines that such treatment is warranted based on the signals detected by the sensing electrodes 1412 and processed by the medical device controller 1420. Example therapy electrodes 1414 can include metal electrodes such as stainless-steel electrodes that include one or more conductive gel deployment devices configured to deliver conductive gel to the metal electrode prior to delivery of a therapeutic shock.

In some implementations, medical devices as described herein can be configured to switch between a therapeutic medical device and a monitoring medical device that is configured to only monitor a patient (e.g., not provide or perform any therapeutic functions). For example, therapeutic components such as the therapy electrodes 1414 and associated circuitry can be optionally decoupled from (or coupled to) or switched out of (or switched in to) the medical device. For example, a medical device can have optional therapeutic elements (e.g., defibrillation and/or pacing electrodes, components, and associated circuitry) that are configured to operate in a therapeutic mode. The optional therapeutic elements can be physically decoupled from the medical device to convert the therapeutic medical device into a monitoring medical device for a specific use (e.g., for operating in a monitoring-only mode) or a patient. Alternatively, the optional therapeutic elements can be deactivated (e.g., via a physical or a software switch), essentially rendering the therapeutic medical device as a monitoring medical device for a specific physiologic purpose or a particular patient. As an example of a software switch, an authorized person can access a protected user interface of the medical device and select a preconfigured option or perform some other user action via the user interface to deactivate the therapeutic elements of the medical device.

FIG. 14B illustrates a hospital wearable defibrillator 1400A that is external, ambulatory, and wearable by a patient 1402. Hospital wearable defibrillator 1400A can be configured in some implementations to provide pacing therapy, e.g., to treat bradycardia, tachycardia, and asystole conditions. The hospital wearable defibrillator 1400A can include one or more ECG sensing electrodes 1412a, one or more therapy electrodes 1414a and 1414b, a medical device controller 1420 and a connection pod 1430. For example, each of these components can be structured and function as like number components of the medical device 1400. For example, the electrodes 1412a, 1414a, 1414b can include disposable adhesive electrodes. For example, the electrodes can include sensing and therapy components disposed on separate sensing and therapy electrode adhesive patches. In some implementations, both sensing and therapy components can be integrated and disposed on a same electrode adhesive patch that is then attached to the patient. For example, the front adhesively attachable therapy electrode 1414a attaches to the front of the patient's torso to deliver pacing or defibrillating therapy. Similarly, the back adhesively attachable therapy electrode 1414b attaches to the back of the patient's torso. In an example scenario, at least three ECG adhesively attachable sensing electrodes 1412a can be attached to at least above the patient's chest near the right arm, above the patient's chest near the left arm, and towards the bottom of the patient's chest in a manner prescribed by a trained professional.

A patient being monitored by a hospital wearable defibrillator and/or pacing device may be confined to a hospital bed or room for a significant amount of time (e.g., 75% or more of the patient's stay in the hospital). As a result, a user interface 1460a can be configured to interact with a user other than the patient, e.g., a nurse, for device-related functions such as initial device baselining, setting and adjusting patient parameters, and changing the device batteries.

In some implementations, an example of a therapeutic medical device that includes a digital front-end in accordance with the systems and methods described herein can include a short-term defibrillator and/or pacing device. For example, such a short-term device can be prescribed by a physician for patients presenting with syncope. A wearable defibrillator can be configured to monitor patients presenting with syncope by, e.g., analyzing the patient's physiological and cardiac activity for aberrant patterns that can indicate abnormal physiological function. For example, such aberrant patterns can occur prior to, during, or after the onset of syncope. In such an example implementation of the short-term wearable defibrillator, the electrode assembly can be adhesively attached to the patient's skin and have a similar configuration as the hospital wearable defibrillator described above in connection with FIG. 14A.

FIGS. 14C and 14D illustrate example wearable patient monitoring devices with no treatment or therapy functions. For example, such devices are configured to monitor one or more physiological parameters of a patient, e.g., for remotely monitoring and/or diagnosing a condition of the patient. For example, such physiological parameters can include a patient's ECG information, tissue (e.g., lung) fluid levels, cardio-vibrations (e.g., using accelerometers or microphones), and other related cardiac information. A cardiac monitoring device is a portable device that the patient can carry around as he or she goes about their daily routine.

Referring to FIG. 14C, an example wearable patient monitoring device 1400C can include tissue fluid monitors 1465 that use radio frequency (RF) based techniques to assess fluid levels and accumulation in a patient's body tissue. Such tissue fluid monitors 1465 can be configured to measure fluid content in the lungs, typically for diagnosis and follow-up of pulmonary edema or lung congestion in heart failure patients. The tissue fluid monitors 1465 can include one or more antennas configured to direct RF waves through a patient's tissue and measure output RF signals in response to the waves that have passed through the tissue. In certain implementations, the output RF signals include parameters indicative of a fluid level in the patient's tissue. In examples, device 1400C may be a cardiac monitoring device that also includes digital sensing electrodes 1470 for sensing ECG activity of the patient. Device 1400C can pre-process the ECG signals via one or more ECG processing and/or conditioning circuits such as an ADC, operational amplifiers, digital filters, signal amplifiers under control of a microprocessor. Device 1400C can transmit information descriptive of the ECG activity and/or tissue fluid levels via a network interface to a remote server for analysis.

Referring to FIG. 14D, another example wearable cardiac monitoring device 1400D can be attached to a patient via at least three adhesive digital cardiac sensing electrodes 1475 disposed about the patient's torso. Cardiac devices 1400C and 1400D are used in cardiac monitoring and telemetry and/or continuous cardiac event monitoring applications, e.g., in patient populations reporting irregular cardiac symptoms and/or conditions. These devices can transmit information descriptive of the ECG activity and/or tissue fluid levels via a network interface to a remote server for analysis. Example cardiac conditions that can be monitored include atrial fibrillation (AF), bradycardia, tachycardia, atrio-ventricular block, Lown-Ganong-Levine syndrome, atrial flutter, sino-atrial node dysfunction, cerebral ischemia, pause(s), and/or heart palpitations. For example, such patients may be prescribed a cardiac monitoring for an extended period of time, e.g., 10 to 30 days, or more. In some ambulatory cardiac monitoring and/or telemetry applications, a portable cardiac monitoring device can be configured to substantially continuously monitor the patient for a cardiac anomaly, and when such an anomaly is detected, the monitor can automatically send data relating to the anomaly to a remote server. The remote server may be located within a 24-hour manned monitoring center, where the data is interpreted by qualified, cardiac-trained reviewers and/or HCPs, and feedback provided to the patient and/or a designated HCP via detailed periodic or event-triggered reports. In certain cardiac event monitoring applications, the cardiac monitoring device is configured to allow the patient to manually press a button on the cardiac monitoring device to report a symptom. For example, a patient can report symptoms such as a skipped beat, shortness of breath, light headedness, racing heartrate, fatigue, fainting, chest discomfort, weakness, dizziness, and/or giddiness. The cardiac monitoring device can record predetermined physiologic parameters of the patient (e.g., ECG information) for a predetermined amount of time (e.g., 1-30 minutes before and 1-30 minutes after a reported symptom). As noted above, the cardiac monitoring device can be configured to monitor physiologic parameters of the patient other than cardiac related parameters. For example, the cardiac monitoring device can be configured to monitor, for example, cardio-vibrational signals (e.g., using accelerometers or microphones), pulmonary-vibrational signals, breath vibrations, sleep related parameters (e.g., snoring, sleep apnea), tissue fluids, among others.

In some examples, the devices described herein (e.g., FIGS. 14A-14D) can communicate with a remote server via an intermediary or gateway device 1480 such as that shown in FIG. 14D. For instance, devices such as shown in FIGS. 14A-D can be configured to include a network interface communications capability as described herein in reference to, for example, FIG. 3.

Additionally, the devices described herein (e.g., FIGS. 14A-14D) can be configured to include one or more accelerometers as described herein. For example, as noted above in the discussion of FIGS. 1A-1C, one or more accelerometers can be integrated into various components of a wearable device or included as a standalone accelerometer configured to measure movement of a patient.

Although the subject matter contained herein has been described in detail for the purpose of illustration, it is to be understood that such detail is solely for that purpose and that the present disclosure is not limited to the disclosed embodiments, but, on the contrary, is intended to cover modifications and equivalent arrangements that are within the scope of the appended claims. For example, it is to be understood that the present disclosure contemplates that, to the extent possible, one or more features of any embodiment can be combined with one or more features of any other embodiment.

Other examples are within the scope of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

1. An ambulatory externally worn cardiac device configured to automatically assess an ambulatory patient's physical condition, the device comprising:
one or more externally worn ECG sensing electrodes configured to detect at least one cardiac electric signal of the ambulatory patient;
one or more motion detectors configured to detect at least one motion signal indicative of patient movement; and
at least one processor operably coupled to the one or more externally worn sensing electrodes and the one or more motion detectors, the at least one processor being configured to
receive, for the ambulatory patient wearing the device, an initiating criterion corresponding to an ambulatory exertion test, wherein the initiating criterion specifies heartrate-dependent parameter requirements and/or activity level-dependent parameter requirements,
continuously monitor a current heartrate-dependent parameter and/or a current activity level-dependent parameter of the ambulatory patient,
periodically determine whether the monitored current heartrate-dependent parameter and/or the current activity level-dependent parameter satisfies the initiating criterion for the ambulatory exertion test,
invite, via a user interface prompt, the ambulatory patient to participate in the ambulatory exertion test, and
initiate, upon receiving patient input indicating acceptance of the invitation, the ambulatory exertion test.

2. The ambulatory externally worn cardiac device of claim 1,
wherein the heartrate-dependent parameter is continuously monitored, and wherein the heartrate-dependent parameter comprises one or more of heartrate, energy exerted by the ambulatory patient, work performed by the ambulatory patient, heartrate variability, premature ventricular contraction burden or counts, atrial fibrillation burden, pauses, heartrate turbulence, QRS height, QRS width, changes in ECG morphology, cosine R-T, QT interval, QT variability, T-wave width, T-wave alternans, T-wave amplitude, T-wave variability, R-wave amplitude, or ST segment changes.

3. The ambulatory externally worn cardiac device of claim 1,
wherein the current activity level-dependent parameter is continuously monitored, and wherein the current activity level-dependent parameter comprises one or more of activity type, activity level, activity duration, energy exerted by the ambulatory patient, or work performed by the ambulatory patient.

4. The ambulatory externally worn cardiac device of claim 1,
wherein the at least one processor is further configured to receive a prescription for the ambulatory exertion test for the ambulatory patient from a healthcare provider associated with the ambulatory patient, the prescription comprising the initiating criterion for the ambulatory exertion test.

5-14. (canceled)

15. The ambulatory externally worn cardiac device of claim 1, the at least one processor being further configured to:
monitor the ambulatory patient during the ambulatory exertion test;
determine if the ambulatory patient is satisfying at least one sustaining criterion during the ambulatory exertion test, the at least one sustaining criterion indicating whether the ambulatory patient is satisfactorily completing the ambulatory exertion test; and
terminate the ambulatory exertion test if the ambulatory patient does not satisfy the at least one sustaining criterion.

16. The ambulatory externally worn cardiac device of claim 15, wherein the at least one sustaining criterion comprises one or more of a minimum level of activity to be maintained by the ambulatory patient during the ambulatory exertion test or a target heart range for the ambulatory patient during the ambulatory exertion test.

17. The ambulatory externally worn cardiac device of claim 1, the at least one processor being further configured to:
monitor the ambulatory patient during the ambulatory exertion test;
determine that the ambulatory patient has satisfied at least one terminating criterion for the ambulatory exertion test;
provide an indication to the ambulatory patient that the ambulatory patient has completed the ambulatory exertion test; and
terminate the ambulatory exertion test.

18. The ambulatory externally worn cardiac device of claim 17, wherein to terminate the ambulatory exertion test comprises to generate a report comprising patient performance information as measured during the ambulatory exertion test.

19-27. (canceled)

28. The ambulatory externally worn cardiac device of claim 1, the at least one processor being further configured to:
administer a plurality of ambulatory exertion tests to the ambulatory patient;
record patient performance information for each of the plurality of ambulatory exertion tests to generate historical patient performance information;
administer a new ambulatory exertion test to the ambulatory patient;
record updated patient performance information for the new ambulatory exertion test;
compare the updated patient performance information to the historical patient performance information to produce comparison exertion test information; and
determine at least one trend for the ambulatory patient based upon the comparison exertion test information, the at least one trend indicating a change in an overall cardiac condition of the ambulatory patient.

29. The ambulatory externally worn cardiac device of claim 1, wherein the ambulatory exertion test comprises one or more of a distance-based walk test, a distance-base run test, a distance-based climb test, a time-base walk test, a time-base run test, or a time-based climb test.

30. The ambulatory externally worn cardiac device of claim 1, wherein the one or more motion detectors comprise one or more accelerometers configured to generate a plurality of motion signals representative of movement of the ambulatory patient.

31. The ambulatory externally worn cardiac device of claim 30, wherein the at least one processor is configured to periodically determine the current activity level-dependent parameter of the ambulatory patient by being further configured to:
analyze the plurality of motion signals to determine if at least a portion of the plurality of motion signals exceed a motion threshold; and
generate a motion classification based on determining that at least a portion of the plurality of motion signals exceed the motion threshold, the motion classification comprising a motion type and a current level of activity of the ambulatory patient.

32. The ambulatory externally worn cardiac device of claim 31, wherein the motion threshold comprises one or more of a change in signal amplitude on at least one directional axis of the plurality of motion signals that exceeds 50% over a period of time, a change in signal on at least one directional axis of the plurality of motion signals that exceeds 100% over a period of time, or a change in signal on at least one directional axis of the plurality of motion signals that exceeds 150% over a period of time.

33. (canceled)

34. A non-transitory computer-readable medium storing computer-executable instructions to implement a process to automatically assess a physical condition of an ambulatory patient wearing an ambulatory externally worn cardiac device, the instructions comprising instructions to:
receive, for the ambulatory patient wearing the device, an initiating criterion corresponding to an ambulatory exertion test, wherein the initiating criterion specifies heartrate-dependent parameter requirements and/or activity level-dependent parameter requirements;
continuously monitor a current heartrate-dependent parameter and/or a current activity level-dependent parameter of the ambulatory patient;
periodically determine whether the monitored current heartrate-dependent parameter and/or the current activity level-dependent parameter satisfies the initiating criterion for the ambulatory exertion test;
invite, via a user interface prompt, the ambulatory patient to participate in the ambulatory exertion test; and
initiate, upon receiving patient input indicating acceptance of the invitation, the ambulatory exertion test.

35-41. (canceled)

42. The non-transitory computer-readable medium of claim 34, wherein the initiating criterion further comprises monitoring the ambulatory patient performing a motion activity for a minimum period of time as determined for the ambulatory patient.

43. The non-transitory computer-readable medium of claim 42, wherein the motion activity comprises one or more of walking, running, or climbing stairs.

44. The non-transitory computer-readable medium of claim 42, wherein the minimum period of time comprises thirty seconds, one minute, two minutes, three minutes, five minutes, or ten minutes.

45. The non-transitory computer-readable medium of claim 42, wherein the ambulatory exertion test comprises a physical test directed to the ambulatory patient and configured to prompt the ambulatory patient to perform a particular activity for a predetermined period of time.

46. The non-transitory computer-readable medium of claim 45, wherein the physical test comprises one or more of a walking test, a jogging test, a running test, or a stair climbing test.

47. The non-transitory computer-readable medium of claim 45, wherein the predetermined period of time comprises at least one of one minute, two minutes, five minutes, six minutes, ten minutes, fifteen minutes, twenty minutes, or thirty minutes.

48-57. (canceled)

58. The non-transitory computer-readable medium of claim 34, the instructions further comprising instructions to:
monitor the ambulatory patient during a baseline ambulatory exertion test; and
determine baseline performance information for the ambulatory patient based upon the baseline ambulatory exertion test.

59. (canceled)

60. The non-transitory computer-readable medium of claim 58, the instructions further comprising instructions to:
generate ambulatory patient performance information for the ambulatory patient upon completion of the ambulatory exertion test;
compare the ambulatory patient performance information to the baseline performance information to produce comparison exertion test information; and
determine at least one trend for the ambulatory patient based upon the comparison exertion test information, the at least one trend indicating a change in an overall cardiac condition of the ambulatory patient.

61-62. (canceled)

63. A method for assessing a physical condition of an ambulatory patient wearing an external cardiac device, the method comprising:
receiving, for the ambulatory patient wearing the external cardiac device, an initiating criterion corresponding to an ambulatory exertion test, wherein the initiating criterion specifies one or more of heartrate dependent parameter requirements or activity level dependent parameter requirements;
continuously monitoring one or more of a current heartrate dependent parameter or a current activity level dependent parameter of the ambulatory patient;
periodically determining whether one or more of the current heartrate dependent parameter or the current activity level dependent parameter satisfies the initiating criterion for the ambulatory exertion test;
presenting, via a user interface prompt, an invitation for the ambulatory patient to participate in the ambulatory exertion test; and
initiating, upon receiving patient input indicating acceptance of the invitation, the ambulatory exertion test.

64. The method of claim 63, further comprising:
monitoring the ambulatory patient during the ambulatory exertion test;
determining if the ambulatory patient is satisfying at least one sustaining criterion during the ambulatory exertion test, the at least one sustaining criterion indicating whether the ambulatory patient is satisfactorily completing the ambulatory exertion test; and
terminating the ambulatory exertion test if the ambulatory patient does not satisfy the at least one sustaining criterion.

65. The method of claim 64, wherein the at least one sustaining criterion comprises one or more of a minimum level of activity to be maintained by the ambulatory patient during the ambulatory exertion test or a target heart range for the ambulatory patient during the ambulatory exertion test.

66. The method of claim 63, further comprising:
monitoring the ambulatory patient during the ambulatory exertion test;
determining that the ambulatory patient has satisfied at least one terminating criterion for the ambulatory exertion test;
providing an indication to the ambulatory patient that the ambulatory patient has completed the ambulatory exertion test; and
terminating the ambulatory exertion test.

67. The method of claim 66, wherein to terminate the ambulatory exertion test comprises to generate a report comprising patient performance information as measured during the ambulatory exertion test.

68. The method of claim 66, wherein the at least one terminating criterion comprises one or more of a total distance walked, a total distance run, a total number of stairs climbed, or a total time elapsed.

69. The method of claim 63, further comprising:
monitoring a cardiac electrical signal and at least one motion signal for the ambulatory patient during the ambulatory exertion test; and
determining patient performance information for the ambulatory patient during the ambulatory exertion test.

70. The method of claim 69, further comprising outputting a performance message to the ambulatory patient during the ambulatory exertion test, the performance message comprising one or more of an indication to increase pace, an indication to decrease pace, an indication to maintain current pace, an indication of time remaining, or an indication of elapsed time.

71. The method of claim 69, further comprising outputting at least a portion of the patient performance information to the ambulatory patient during the ambulatory exertion test, the patient performance information comprising one or more of total steps taken during the ambulatory exertion test, total distance covered during the ambulatory exertion test, total stairs climbed or descended during the ambulatory exertion test, average heartrate during the ambulatory exertion test, maximum heartrate during the ambulatory exertion test, or minimum heartrate during the ambulatory exertion test.

72. The method of claim 63, further comprising:
monitoring cardiac activity for the ambulatory patient during the ambulatory exertion test based upon a cardiac electric signal;
determining that the ambulatory patient is experiencing or has experienced cardiac arrhythmia during the ambulatory exertion test; and
terminating the ambulatory exertion test upon determining that the ambulatory patient is experiencing or has experienced cardiac arrhythmia during the ambulatory exertion test.

73. The method of claim 63, further comprising:
monitoring current motion activity for the ambulatory patient during the ambulatory exertion test based upon at least one motion signal;
determining that the ambulatory patient is experiencing or has experienced a fall event during the ambulatory exertion test; and
terminating the ambulatory exertion test upon determining that the ambulatory patient is experiencing or has experienced the fall event during the ambulatory exertion test.
